(19) United States
(12) Patent Application Publication
(10) Pub. No.: US 2012/0330116 A1
(43) Pub. Date: Dec. 27, 2012

(54) System for Non-Invasive Assay of Liver Function

(75) Inventors: Philip E. Eggers, Dublin, OH (US); Eric A. Eggers, Portland, OR (US); Andrew R. Eggers, Ostrander, OH (US); Mark A. Mayerchak, Bothell, WA (US)

(73) Assignee: CARDIOX CORPORATION, Columbus, OH (US)

(21) Appl. No.: 13/597,999

(22) Filed: Aug. 29, 2012

Related U.S. Application Data
(63) Continuation-in-part of application No. 12/754,888, filed on Apr. 6, 2010.
Application No. 12/754,888 is a continuation-in-part of application No. 12/418,866, filed on Apr. 6, 2009.
(60) Provisional application No. 61/528,562, filed on Aug. 29, 2011.

(51) Int. Cl. A61B 5/1455

(52) U.S. Cl. 600/314; 600/317

(57) ABSTRACT

A system, method and apparatus are disclosed for using a transcutaneous detection system to measure the quantity of a circulating organ activity detection analyte in the blood, and thereby assay the activity of an organ. A preferred organ for assay is the human liver and a preferred indicator is indocyanine green (ICG) dye The procedure is under the control of a monitor/controller having a visual display and capable of providing cues to the operator. A sensor array apparatus for use in conjunction with the system monitor/controller is configured for increased sensitivity of assaying organ function.

### CROSS-REFERENCE TO RELATED APPLICATIONS

This application claims priority to pending U.S. Provisional Patent Application Ser. No. 61/528,562, filed Aug. 29, 2011, entitled “System for Non-Invasive Quantification of Liver Function” and claims priority to pending U.S. patent application Ser. No. 12/754,888, filed Apr. 6, 2010, entitled “Hemodynamic Detection of Circulatory Anomalies,” which is a continuation in part of and also claims priority to U.S. patent application Ser. No. 12/418,866, filed Apr. 6, 2009 and entitled “Hemodynamic Detection of Circulatory Anomalies,” the disclosures of which are incorporated by reference. The application also qualifies as an application under 35 U.S.C. §371 and claims the benefit of to pending PCT application No. PCT/US11/31433, filed Apr. 6, 2011 and published in English under PCT Article 21(2) the disclosure of which is also incorporated by reference.

### STATEMENT REGARDING FEDERALLY SPONSORED RESEARCH

Not applicable.

### BACKGROUND

The present invention generally relates to a system, method and apparatus for measuring the activity of the liver in a patient. A fluorescent analyte is introduced into the circulatory system of the patient and the concentration of the analyte is measured over time. The fluorescence emissions and detection system disclosed can also be used to measure the blood concentration of a compound that is metabolized by the liver or other organs, or excreted. Thus, the activity of liver enzymes, liver circulatory capacity, and liver function, along with the function of other organs can be measured with the appropriate detection compound.

In addition to using percutaneous or invasive detection systems to monitor cardiac activity and circulation, systems are needed to rapidly, continuously and repeatedly measure the concentration of agents introduced into the blood that are indicative of the level of organ function. In particular, the ability of the liver to metabolize a detection agent has been employed to measure the level of activity of the liver in patients. Patients suffering from trauma, sepsis and hepatitis may have their liver function evaluated (i.e., the level of activity of the liver organ for metabolizing monitoring agents) to determine if the liver has been compromised. In addition, patients suffering from liver cirrhosis or patients in need of a liver transplant, or post-operative liver transplant patients require liver function assays in order to appropriately guide treatment regimes.

Similarly, the relative ability of other organs, such as the kidney, to metabolize circulating substances would be useful in planning and monitoring treatment for conditions affecting those organs.

Existing systems for tracking the movement through circulation, or the level of continued presence of a tracking agent suffer from low resolution and reproducibility in the clinical setting. For instance, a continuing difficulty with existing methods for detection of cardiac anomalies is the efficacy of using microbubbles as a circulatory tracking indicator. Several existing methods for analyzing cardiac, pulmonary and general circulation including transesophageal echocardiography, transthoracic echocardiography, and the transcranial Doppler method, suffer from barriers for routine use for screening, whether due to the need for anesthesia or expensive equipment. There is a need for more efficient circulatory tracking reagents, i.e. a reagent that can be reproducibly introduced into the circulatory system, be quantitatively detectable, and utilize relatively straightforward detection systems that are easily tolerated by patients.

In addition there's a long history of utilization of natural and labeled compounds to assay the activity of the liver by correlating the liver's capacity to remove plasma-borne compounds from circulation. See, e.g., Korman, et al., NEJM 292:1205 (1975) and Horak, et el., Gastronterology. 71: 809 (1976).

Mills, in U.S. Pat. No. 6,030,841 discloses a number of compounds, including fluorescent compounds that can be utilized for liver function assays. Mills identifies a variety of fluorescently labeled compounds, radio-labeled compounds, and colorimetric assays. A large family of substituted steroid fluorescent compounds, including bile acid derivatives are disclosed for utilization in assays of liver function. It is noteworthy that the state of the art method of assay disclosed in Mills involves repeated blood draws, plasma processing and a dedicated fluorescence spectrophotometer (e.g., Perkin Elmer LS5B Spectrometer).

Wissler noted that the elimination of an analyte such as ICG from the circulating blood by the liver behaves as a two-compartment system. The dye injected into the blood forms a decreasing reservoir in the systemic plasma, with a large percentage of the dye being removed after passage through the liver. A smaller percentage is recirculating through the hepatic vein into the systemic plasma. The liver effectively acts as a second compartment, accumulating sequestered dye, with the liver sequestered dye being excreted into the bile at a rate (consummate) with the processing capacity of a particular liver. See Wissler, E. H., E. J. Appl. Physiol. 111: 641-646 (2011). A number of liver activity factors are important during clinical evaluation of a patient. Such factors include the rate at which a patient's liver extracts dye from the systemic plasma, and the rate at which the liver excretes sequestered due into the bile.

Indocyanine green is a well-characterized dye and is widely used as a fluorescent indicator. ICG has been marketed as a lyophilized powder (Akorn, Inc. Buffalo Grove, Ill.). ICG has been used for a number of diagnostic procedures, including for angiography and ophthalmology, and also for procedures such as dye enhanced photocoagulation, and photodynamic therapy. ICG has a long history in clinical settings, is well tolerated and can be utilized at relatively high plasma concentrations without significant side effects. ICG has been approved by the U.S. FDA as an injectable drug for ophthalmic angiography, measuring cardiac output, liver blood flow and liver function.

Because of its long history and general acceptance, a number of ICG analogs have been developed. Alam, et al. discloses enhanced compositions of ICG for use in diagnostic and therapeutic procedures. Alam, et al. US Parent Application Publication US 2003/0060718 (2003). The Alam, et al., publication does not disclose or reference apparatus for performing such procedures, but does claim a method for performing angiography (see claims 52-66) or treating lesions such as tumors (see, e.g., claims 66, 81). The claims presented in the Alam, et al., application do not present any impediment to the currently projected practice of the Cardiox system or contemplated extensions. Moreover, the ICG compositions disclosed by Alam, et al., could be utilized with the Cardiox system if the composition was purchased by a licensed supplier.

With respect to monitoring liver function using a circulating tracking agent, both invasive and minimally invasive systems exist, but have not been widely accepted or are ripe for improvement. Invasive systems rely on the injection of a dye, followed by withdrawing a blood sample and spectrophotometric analysis of the sample at regular intervals. Such a system is hampered by its labor-intensive nature, and the errors introduced by repeated inexact manual steps. A related transcutaneous system uses the injection of a dye, and then the dye concentration is measured by pulsed-light densitometry using a transcutaneous detector. The existing transcutaneous system requires a relatively high dye dosage (i.e., 20-50 mg ICG per test) in order to allow detection. Such high doses preclude continuous monitoring of liver function because the highest allowable daily dose of ICG (about 80-90 mg/day) is quickly exceeded. For additional background, see, “Indocyanine green elimination rate detects hepatocellular dysfunction early in septic shock and correlates with survival. Crit Care Med. 29:1159-63, 2001; and Sakka S, et al: “Prognostic Value of the Indocyanine Green Plasma Disappearance Rate in Critically III Patients.” Chest 122: 1715-20, 2002.

A number of public domain systems exist that utilize a single irradiation source, such as a fiber optic cable connected to a laser and an associated separate detector, such as a CCD camera or spectrophotometer. The laser emitter and fluorescence detector have been further associated with endoscopic devices. Even simpler densitometric systems have likewise existed for some time, with such systems relying on the absorption of ICG in the 805-810 nm range. An ear densitometer produced by the Waters Co. of Rochester, Minn. is advertised as being capable of in vivo measurement of ICG absorbance, but in the aforementioned higher doses.

In light of the marginally useful devices available for continuous monitoring of liver or other organ function using a circulating analyte, the present disclosure provides the advantages of a useful non-invasive system. Thus, in application for U.S. patent Ser. No. 12/418,866, to which priority is claimed, a generally non-invasive technique for screening for measuring the concentration of a circulating analyte is disclosed. The present disclosure provides advantages that will be apparent. This results in one or more intensity versus time curves, representing an analyte concentration resulting from metabolism of the indicator through the circulatory system, i.e. by the lungs, brain, kidney or other organs.

Implementation of the transcutaneous detection system for accurate detection of a circulating indicator allows monitoring of the decay of a circulating indicator in the blood, and thus allows for monitoring of organ function with relatively low doses of indicator.

### BRIEF SUMMARY

The present system discloses using a transcutaneous detection system to measure the quantity of a circulating detection agent in the blood, and thereby measure the decay of concentration due to liver function. The present system utilizes a variation of the previously disclosed system, method and apparatus for detecting and quantifying right-to-left pulmonary shunts. The preferred indicator, which is employed, is indocyanine green (ICG) dye, which will fluoresce when exposed to an appropriate wavelength of higher energy light, for example, a laser in the near infrared region. The procedure is under the control of a monitor/controller having a visual display and capable of providing a cue to the operator. A vein access catheter is employed in connection with a peripheral vein such as the antecubital vein in an arm. Sensing of the indicator concentration takes place at an arterial vasculature of the animal body, preferably at the pinna or scaphoid fossa of the human ear or the finger of the hand.

The system is embodied preferably to perform assays using fluorescence sensor arrays each with three indicator fluorescing lasers, which are directed to a blood vessel under the skin surface at a location where relatively thin tissue contains a blood vessel network. These sensors are configured for transmission mode measurement wherein three lasers are combined with aspheric collimating lenses positioned opposite a photon collimating orifice and an optical band pass filter, selected to enhance selective passage of fluorescing photons to a photodetector while greatly limiting the incidence of the excitation photons at the photodetector. The two branches of these fluorescence sensor array configurations are preferably spring biased, adjustable or have fixed size gap opening (“throat”) to be held in proper and stable positions on accessible tissue.

The monitor/controller may be configured to calculate the concentration of a blood-borne indicator relative to baseline (e.g., in units of millivolts of measured fluorescence signal level), and as the indicator is metabolized the liver function can be determined by measuring the relative rate of disappearance of the indicator from the blood stream. Similarly the system can be used to calculate indicator/analyte sequestration or elimination at a steady state.

Using the data collected by the system, the monitor/controller publishes the relative indicator concentration decay as a function of time curves and the derived exponential decay coefficient, plasma disappearance rate, and residual relative concentration at 15 minutes after injection of the indicator.

In a further embodiment, the system implements comprises a sensor array with transmission mode sensing in which the sensor array comprises two or more pairs of emitters of excitation photons and fluorescence detectors of the fluorescent analyte of liver activity. The several emitters of excitation photons and fluorescence detectors are optionally energizable in a sequence of such emitter detector pairs or energizable simultaneously, wherein the monitor/controller is responsive to elect one or more of that pair exhibiting an average detection signal output of highest intensity.

A further embodiment is a sensor array apparatus in which the light path of excitation photons is arranged with an aspheric collimating lens, and the light path of emitted fluorescent photons to the photodetector is arranged with a collimator plate and an interference filter. A preferred embodiment is where the excitation source emits photons in a wavelength range of from about 750 to 820 nanometers, or even more preferably at a wavelength of from about 780 to 790 nanometers. The sensor array apparatus may be optionally positioned at paired distal locations by providing two fluorescence sensing array fixtures with sensing array arms, removeably attached to a headband.

A preferred embodiment of the system and apparatus configured for measuring a relative organ activity in a patient, comprises of providing an indicator analyte delivery system having an outlet located in a vein of the patient in blood flow communication with the right side of the heart; said indicator analyte delivery system being actuateable to cue the injection of a fluorescing biocompatible dye excitable by tissue penetrating excitation radiation to derive fluorescence emission corresponding with the indicator analyte concentration; the indicator delivery system includes a flow sensor responsive to derive signals corresponding with the commencement and termination of fluid flow through the system; providing an indicator analyte that is fluorescently labeled and the concentration of said indicator analyte is responsive to the metabolic status of said organ; a sensor array comprising a excitation photon emitter energizable to generate light at the excitation radiation wavelength and a photodetector which is filtered for response substantially only to the fluorescence emission, further providing a transmissive sensor positionable to sense the presence of at least a portion of the indicator at the vasculature of one or of symmetrically paired distal locations of the patient and having one or more outputs corresponding with the instantaneous concentration of indicator at such vasculature in which the sensor array further two or more paired excitation photons emitters and photodetectors and energizable in a sequence of such pairs or simultaneously; even further providing a sensor array further comprising the excitation photon emitters and photodetectors arranged with filtering system comprising an aspheric collimating lens, a collimator plate and an interference filter in the transmission path to the photodetector, and then providing a monitor/controller having a display and responsive to said actuation of the indicator analyte delivery system to commence timing the time following first detection of indicator analyte, responsive to a sensor output; and finally providing an associated monitor/controller responsive to publish one or more of a decay curves or to display one or more indicator analyte decay curves to determine relative activity of the organ, whereby the injection of the indicator analyte commences activation of the excitation photon emitters, and detection of any data signal by the associated photodetectors, with the monitor controller collecting said data signal and calculating a organ function metric.

The disclosure is further embodied in a kit supplying consumable materials necessary for quantifying liver function comprising an indicator delivery tubing system providing a valve, syringe connectors, a flow sensor and sterile intravenous injector, one or more doses of liver activity indicator reagent as a shelf stable material, a diluent for preparing the dose of liver activity indicator reagent for injection or for delivering an indicator bolus and a dose of nonreactive blood compatible clearing reagent for completing the injection. The kit may further be embodied in a flow initiation sensor that further comprises an initiation sensor with a circuit that in communication with a monitor-controller responds to a query determining the number of injections a flow initiation sensor has cued and that is disabled for repeated use after a testing procedure time period. The kit further comprises a sealed tray containing the kit contents maintained in a sterile condition until opened.

Yet another embodiment of the disclosure is a method for measuring the relative concentration over time of a liver activity indicator in the circulating blood stream, comprising the steps a) selecting a liver activity indicator analyte that is predominantly removed from circulating blood by the liver said analyte further comprising a fluorescent moiety that can be excited to emit fluorescence through irradiation by excitation photons in wavelength range known to induce fluorescence in said analyte; b) positioning about the skin of a human subject one or more emitter/detector arrays for emitting excitation photons in a given wavelength range known to induce fluorescence in said fluorescent moiety of the liver activity indicator analyte, said emitter in alignment with one or more detectors configured to measure the intensity of emitted fluorescence photons from the liver activity indicator analyte; c) injecting said liver activity indicator analyte into the blood stream of a human subject; d) recording the relative concentration over time of the liver activity indicator analyte by recording time-varying fluorescence signal level; and e) calculating a liver activity metric according to Formula 1, whereby the relative liver activity of the patient is displayed in a report.

Other objects of the invention will, in part, be obvious and will, in part, appear hereinafter. The various embodiments of the invention, accordingly, comprises the method, apparatus and system possessing the construction, combination of elements, arrangement of parts and steps which are exemplified in the following detailed description.

### BRIEF DESCRIPTION OF THE DRAWINGS

For a full understanding of the nature and objects of the various embodiments of the invention, reference should be had to the following detailed description taken in connection with the accompanying drawings, in which:

FIG. 1 is a schematic perspective view of a patient being tested with the disclosed system;

FIG. 2 is a top view of an indicator delivery system;

FIG. 3A-D are various perspective views of a monitor/controller, which may be used with the disclosed system;

FIG. 4A-G illustrate the structure of a fixed jaw type fluorescent sensor array intended for use with tissue and arterial structure of moderate thickness such as the scaphoid fossa of the ear as shown in FIG. 8;

FIG. 5 is a schematic representation of the alignment orientations of three fluorescence generating and sensing devices utilized with a device of FIG. 4;

FIG. 6 is a perspective view of the cable connector for use with two sensing arrays;

FIG. 7 is a rear view of a human patient wearing the headband apparatus to support sensor arrays about the ears, as shown in FIG. 1;

FIG. 8 is a schematic view of a human ear showing arterial structure at the scaphoid fossa of an ear;

FIG. 9 is a schematic sectional view of fluorescent excitation and detection at the ear of FIG. 8 utilizing a transmission mode detector system;

FIGS. 10A-10C combine as labeled thereon to show a flow chart of the procedure associated with a preferred embodiment;

FIG. 11 is a chart describing a protocol as utilized with a preferred embodiment of the present disclosure;

FIG. 12 shows hypothetical fluorescence emission curves using transmission mode of a single indicator injection for measuring liver activity;

FIG. 13 is a perspective view of the disposable kit components of the apparatus and system;

FIG. 14A-D shows views of a fluid flow detector utilized in a delivery system;

FIG. 15 is a perspective view of the configuration of a flow detection system;

FIG. 16 is a schematic sectional view of fluorescent excitation and detection in tissue when utilizing an array of two or more transmission mode detectors;

### DETAILED DESCRIPTION

A generally applicable non-invasive technique for screening for and measuring the concentration of a circulating blood analyte indicating organ function is disclosed. With the system and method, the analyte is preferably an injectable fluorescing indicator (such as indocyanine green dye). A resultant dilution curve is detected at the vasculature at the scaphoid fossa of the ear, or other chosen location. In general, a near infrared wavelength region laser beam is applied at the ear surface in alternatively a reflection operational mode or a transmissive mode, the transmitted photons are filtered and the fluorescence photons measured for intensity. This results in a curve which is characterized by the exponential decay of the indicator concentration in the blood stream measured during the period beginning about two to three minutes after the time of indicator injection until about 10 to 20 minutes after the time of indicator injection. The starting time for indicator concentration level is delayed to ensure that the injected ICG dye is uniformly mixed throughout the circulating blood volume.

Implementation of the transcutaneous detection system for accurate detection of a circulating indicator allows for monitoring of the decay of a circulating indicator in the blood, and thus allows for monitoring of liver function with relatively low doses of indicator.

The discourse to follow tracks further animal and initial human testing and presents a review of published research, resulting in a diagnostic approach which permits a practical survey for the phenomena over a large patient population.

In general, the preferred embodiments of the present disclosure observe that an indicator such as an externally detectable indicator dye material will traverse through the arterio-venous system from an injection point in a vein, toward the heart. Venous blood containing such an indicator will pass through the heart and the indicator is then carried through the pulmonary circulatory system (i.e., through the lungs) back through the heart and through the various arteries to the tissues of the body. The organs of the body will interact with a circulating indicator, and so long as the presence of the indicator or the metabolic products of the indicator remain in the circulatory system, those indicators and metabolites are detectable by an external or minimally invasive sensor system. The present system is especially adapted to the use of labeled indicators, particularly fluorescently labeled indicators, whose concentration is affected by the metabolic activity of one or more organ. Thus, as organs interact with circulating blood, a blood-borne indicator is subject to degradation, metabolism, excretion, sequestration and other processes to alter the concentration of a measurable indicator in the blood. Because the “indicator” may be modified by organ systems and thus change the detectability of the indicator, as used herein, the indicator is recognized to be the indicator as injected and further modified or metabolized. As described herein an “analyte” is the native indicator dye, and the detectable metabolic products of the indicator, as differentiated from undetectable metabolic products. Thus, an analyte includes a moiety that is measured by the system, directly or indirectly, including products that are analyzed to determine organ activity.

A number of organ systems are subject to analysis by a circulating analyte. In particular, the liver is known to exclusively extract indocyanine green. Thus, the health, vitality, or relative activity of the human liver can be monitored by the liver's capacity to extract indocyanine green from the circulating blood and other tissues of the human body. As noted by Mandell, the ICG dye is rapidly extracted from the blood only by the liver and is excreted in the bile. Mandell Anesth. Analg. 95: 1182-1184 (2002). Analytes that can be used to assist in monitoring other organ systems are known, or could be determined by using the testing systems disclosed herein. Other indicator analytes may be metabolized by the patient body, i.e., modified, excreted, or sequestered. Thus, metabolism may function to remove a detectable form, or to generate a detectable form of an analyte that can be used to monitor various metabolic activities and organ function. Organs adapted for monitoring by the present system include also the kidneys, pancreas, lungs, colon, the immune system, and the brain. Monitoring of organs is primarily limited by the identification of appropriate analytes for use with the system.

A number of fluorescent labeling systems are available for utilizing metabolites as analytes with the present system, including, e.g., labeled alanine aminotransferase (ALT), aspartate aminotransferase (AST), alkaline phosphatase, 5′ nucleotidase and gamma-glutamyl transpeptidase (GGT). With respect to liver function testing, the system disclosed is useful for liver function testing including indications where said testing may be performed in a hospital, outpatient or office-based setting depending on the indication and the condition of the patient. Examples include critically ill patients, especially those with sepsis, acute liver or multi-organ failure, and after multiple trauma; patients with chronically reduced hepatic function (hepatitis, liver cirrhosis); for the evaluation of liver function in organ donors and projected recipients; for monitoring of liver function during liver or abdominal surgery (resection, porto-caval shunt); for diagnosis and monitoring of congenital liver failure in children and neonates; and for the assessment of new drugs and their potential for adverse effect(s) on the liver. Similar parameters are envisioned for using the system with analytes that monitor other organ systems.

As an initial illustration of the system disclosed, a preferred embodiment is the use of indocyanine green (ICG) as a liver activity analyte. Injection of ICG in a vein will lead to the transit of the dye analyte through the circulating blood including to the hepatic artery. As the indicator passes through the liver, a liver extractable indicator such as ICG will be reduced in concentration by the activity of liver enzymes, for instance. Thus, the level of liver activity can be measured by the rate at which the liver activity indicator dye is decreased over time. A variety of metrics can be tested using the present system, including analyte retention rate or residual analyte concentration, R15 at 15.0 minutes after ICG injection (as a percentage); exponential decay coefficient associated with the rate of extraction of the ICG by the liver, K (in reciprocal minutes) and the plasma disappearance rate of ICG in percent per minute.

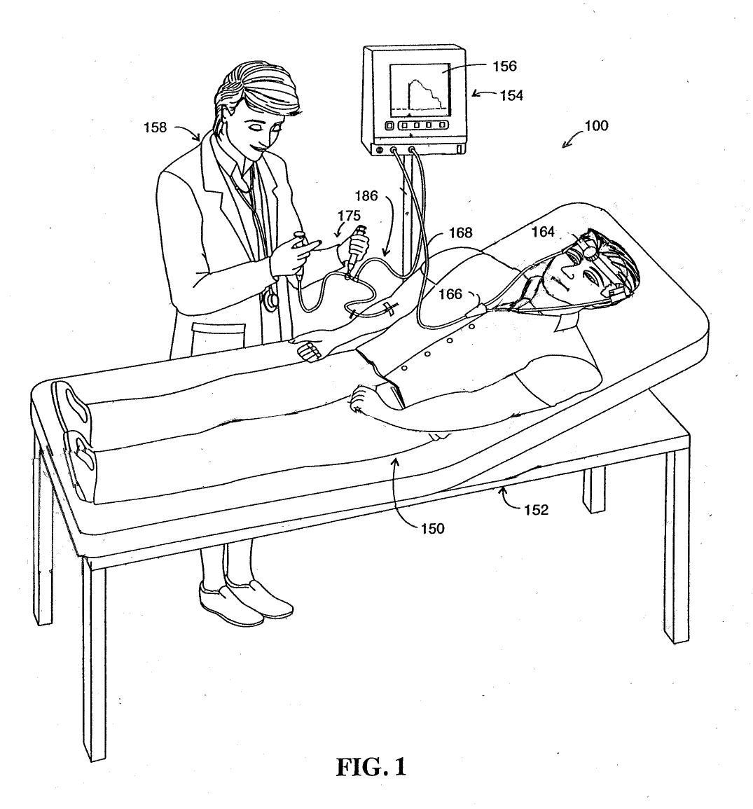

Turning to FIG. 1, a stylized representation of the present system 100 is presented. In the figure, a patient is shown in general at 150 reclining upon an examination bench represented generally at 152. Patient 150 is shown as supine with the head and trunk elevated about 30°. Note that generally, patients undergoing liver monitoring will be monitored in a clinical setting, particularly in a hospital where trauma or disease may be challenging liver function. In out-patient situations for mobile patients, the patient may alternatively be monitored in a sitting position. The monitor is shown in general at 154 having a display 156 which can be observed by the practitioner represented generally at 158. The display could also be coupled to a print apparatus for creating permanent hard copy records of the test parameters and results.

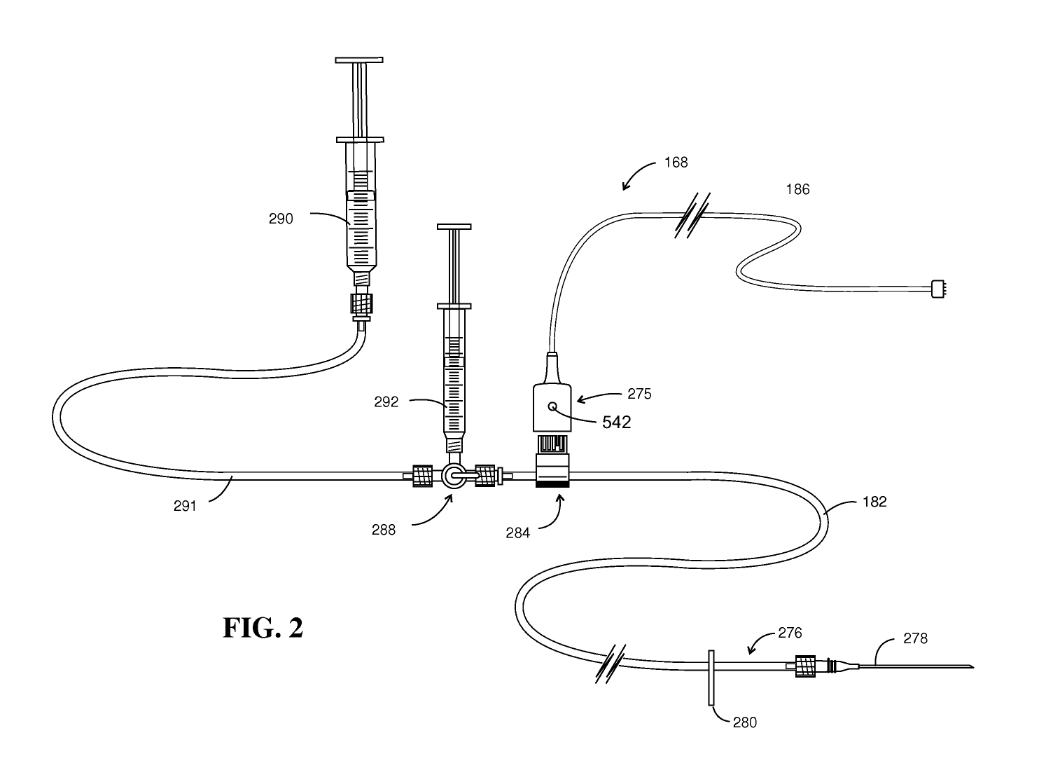

As shown in FIG. 1, the patient is being monitored by sensor arrays about the head. As such patient 150 is wearing a headband 164 supporting a fluorescent sensing array about both ears and in particular the scaphoid fossa of the ear pinna. As discussed in connection with FIG. 7, the sensor arrays are connected with the head support apparatus. As shown, two sensor arrays are in use, with such signals from the two arrays collected at hub 166 and are directed by cable 168 to the monitor 154. Practitioner 158 is holding the injection equipment described in conjunction with FIG. 2, as is illustrated in general at 175 with a cable 186 providing an indicator flow signal to monitor 154. The catheter arrangement 175 is shown in the instant figure having been inserted within the antecubital vein in the right arm of patient 150.

Following injection of a quantity of analyte, (e.g., 5 to 10 mg of the liver activity dye, ICG), a dwell period of about 2 to 3 minutes is allowed to pass. During this dwell period, the indicator is being distributed throughout the circulating blood of the patient, by successive passages through the heart and aorta. After about 2 to 3 minutes, monitoring of the concentration of the liver activity indicator dye can yield a useful signal. It is anticipated that monitoring can be continuous, or can be taken at regular intervals, such as at five-minute intervals. In a preferred embodiment, after injection of the dye, the first measurement is taken at about two minutes post-injection, and then followed by successive measurements at 5 second intervals, for a testing period of about 10 to 20 minutes.

The monitor 154 is configured to calculate the detected relative blood concentration of liver activity indicator analyte (i.e., the detected fluorescence signal level relative to baseline), and provide a semi-logarithmic graph of relative ICG concentration as a function of a linear abscissa, time, as well as the aforementioned metrics exponential decay coefficient, plasma disappearance rate and residual level of ICG at 15 minutes. The slope of the liver activity semi-logarithmic graph indicates the level of enzymatic activity of the liver for a given period of time. For certain patients, the initial monitoring period will confirm adequate liver activity. Other patients may need nearly continuous monitoring, utilizing repeated injections of indicator dye or alternatively, a low continuous dose supplied through an intravenous drip. In addition, certain patients may be subjected to daily monitoring, for instance, and the relative liver activity can be compared over a period of time, including months or weeks.

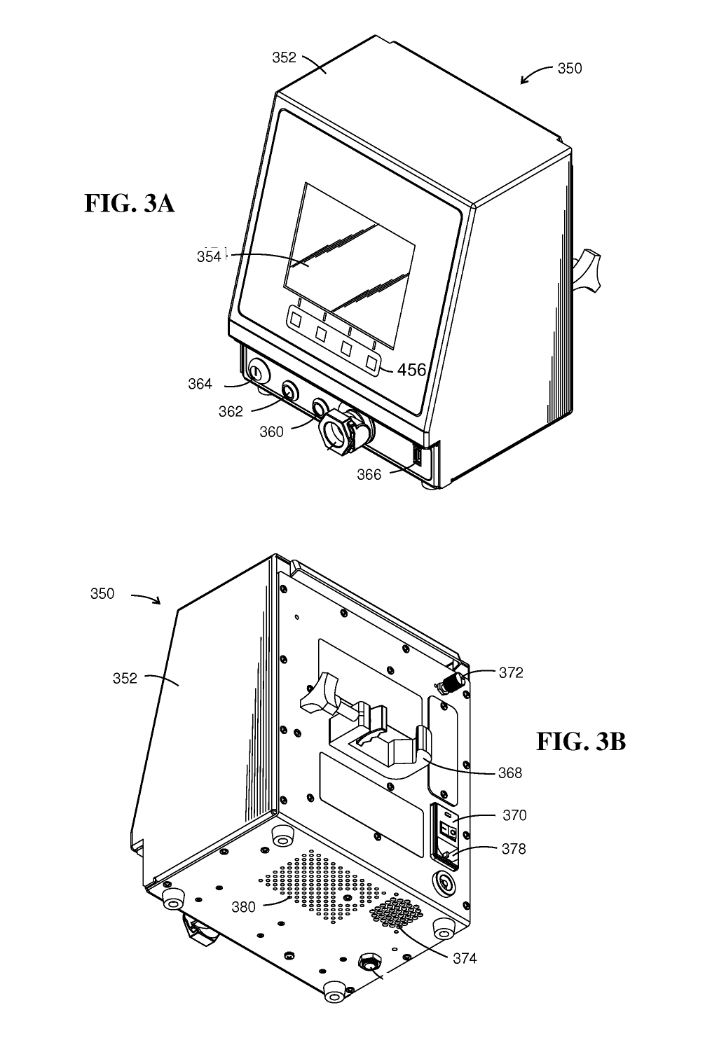

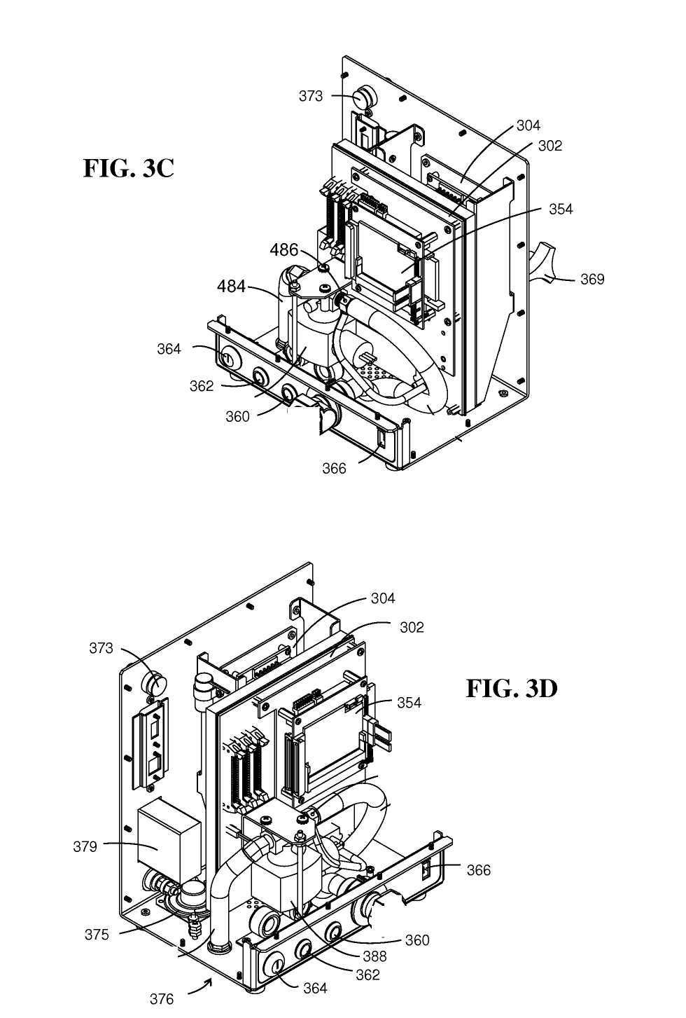

In the succeeding figures, FIGS. 2-9, the general outline of the system components is disclosed. Additional details regarding the components, along with certain alternative embodiments are shown in the examples that follow. In conjunction with the inputs to the system monitor the present procedure incorporates visual and oral cueing in connection with display 154. Proper set-up of the machine, placement of a vein access catheter in a peripheral vein; positioning of the sensor array(s) and introduction of the indicator can be directed and confirmed by the monitor controller. FIG. 2 illustrates one preferred dye indicator delivery mechanism, comparable to that in FIG. 1, that is capable of delivering a single indicator bolus to the patient. Looking to the figure, such equipment is illustrated in general at 168. Equipment 168 includes a relatively short catheter with a 20 gauge needle as represented in general at 276, the needle being shown at 278 and a connector to main tubing being represented at 280. The principal tubing is shown at 182, a flexible elongate delivery tube extending between proximal and distal ends, with an auxiliary catheter coupled in fluid transfer relationship with the distal end defining the outlet. An indicator fluid flow detector represented generally at 284 is coupled in fluid transfer relationship with the proximal end, deriving signals corresponding with the commencement and termination of fluid flow through the system. The indicator flow detector has an output signal at a cable 186 represented in general as ending with flow detector connector 275. Just upstream of flow detector 284 is a 3-way valve represented in general at 288. Connected to valve 288 is a first syringe 292, containing indocyanine green dye (ICG), which initially is injected into the principal tubing 182. Following such injection, the valve 288 is switched and saline solution from a second syringe 290 is injected to, in effect, push the ICG into the antecubical vein. Flow detector 284 detects the dye flow and provides a corresponding signal to the monitor at input 360 (see FIG. 3A). It is from this signal that the monitor determines the commencement of organ analyte introduction.

FIG. 3 provides additional detail concerning the monitor controller for use with the system as represented in general at 350. FIG. 3A shows a front perspective view and FIG. 3B shows a back perspective view of the external features of the monitor/controller, while FIG. 3C shows a right rear perspective view and FIG. 3D shows a left rear perspective view of the internal features of the monitor/controller. The monitor 350 may be mounted on a pole, e.g., an IV pole, includes a housing 352 which provides a display 354 which performs in conjunction with touch switches shown as an array represented generally at 356. Input 360 receives an injection flow signal from cable 186. Adjacent to input 360 is input 362, which receives the signals from the sensor arrays, with input 362 coupled with the earlier described main cable 168 extending from hub connector 166. The lasers are enabled with a key-actuated switch 364 and a flash drive recorder may be received via the USB or other comparable communications port at slot 366. Looking to the rear view at FIG. 3B, the housing 352 may be pole mounted using C-type and shaped clamp 368. Electrical power input and the switching thereof is provided at switch receptacle 370. Audio cue volume potentiometer 372 is used to control the volume of these cues and prompts, and a perforated speaker outlet is provided at 374. The vent 380 adjacent to the speaker outlet provides for system cooling. Looking to the rear view at FIG. 3C and 3D, are perspective views of the interior of the monitor/controller.

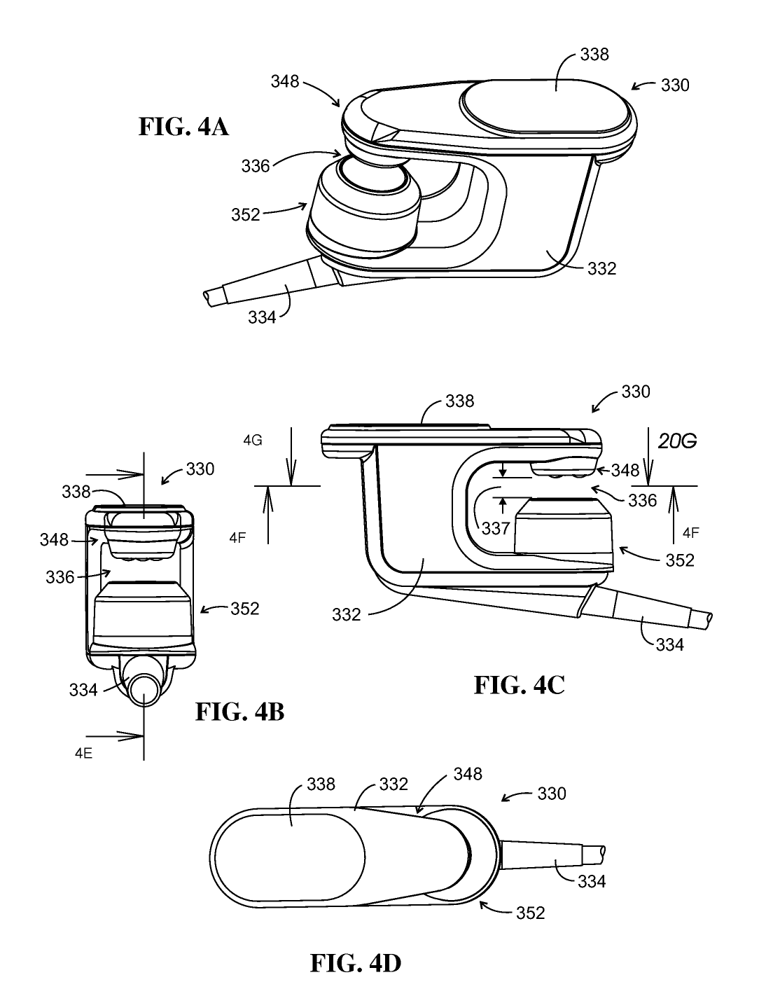

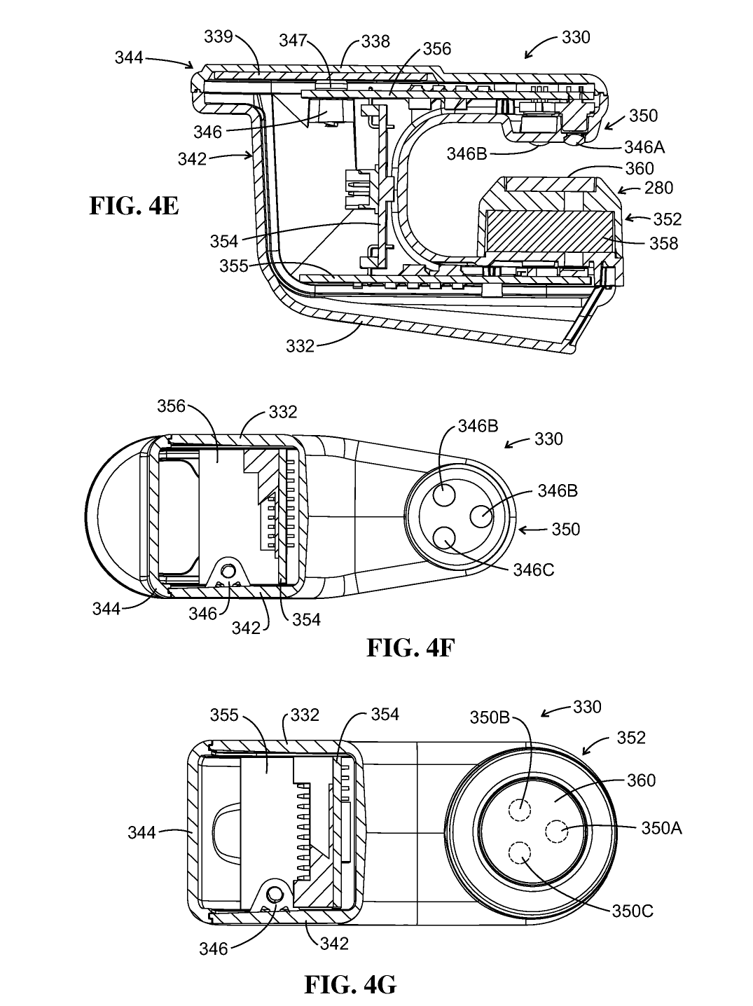

Turning now to FIG. 4, one embodiment of the sensor array is shown that provides for a fixed throat size. Such an embodiment is suitable for placement on most human scaphoid fossa at the ear. The sensing array fixture is shown at 330 in FIG. 4A-4G. FIG. 4A shows a perspective view of array fixture 330, with array fixture 330 being formed of array body 332 with a three laser emitter array support 348 which is integrally connected to a photodiode array detector support 352. The sensor array is connected to the monitor/controller though cable 334. The spaced apart emitter and detector arrays are separated by sensor throat 336, and plate 338 is used to connect the array to a support system. Said features are also shown in relation to the front view of fixture 330 in FIG. 4B, and with respect to side view FIG. 4C. FIG. 4C demonstrates the configuration of throat 336, with the throat opening shown as 337. A top view of the sensor array fixture is in FIG. 4D. FIG. 4E is a longitudinal cross-section of the fixture 330 along plane 4E of FIG. 4B. Contact plate 338 is used to connect the array to a support system, and plate 338 is shown as subtended by magnet 339 or alternatively be formed of ferrous material for attachment to a magnet system. It is also practical to alternatively incorporate a Velcro-type pad or other attachment for the support of device array fixture 330.

Array body 332 is formed of two parts, main body 342 and body cap 344. Cap 344 is retained by press fit, adhesive, or by lug 346 capturing pin 347. Inside the body 332 are found connector board 354, detector board 355, and emitter board 356. A three-laser array and collimating aspheric lenses 346A-C are mounted within a protrusion/emitter head 350 extending outwardly from support body 332. That protrusion is seen, particularly, at FIGS. 4B, 4C and 4F. Complementing the three-laser array is an aligned array of three photodiodes detectors 350A-C located within protrusion 352 which also is seen in FIGS. 4B, 4C and 4G. The cross section in FIG. 4E shows window 360, collimating plate 280, and interference filter 358.

Looking to FIG. 4F, a sectional view is shown through the plane identified at 4F-4F in FIG. 4C. Protrusion 350 is seen to support an array of three lasers and associated aspheric collimating lenses as represented at 346A-346C, aligned with those laser and collimating lenses, as are corresponding photodiodes along with an associated collimator and interference filter. The collimator openings for orifices are seen at FIG. 4G at 350A-350C. Looking to FIG. 4G, a sectional view is shown through the plane identified at 4G-4G in FIG. 4C. The figure shows that a circuit board 355 is mounted in body 344, with connector board 354, body 344 supporting detector array 352.

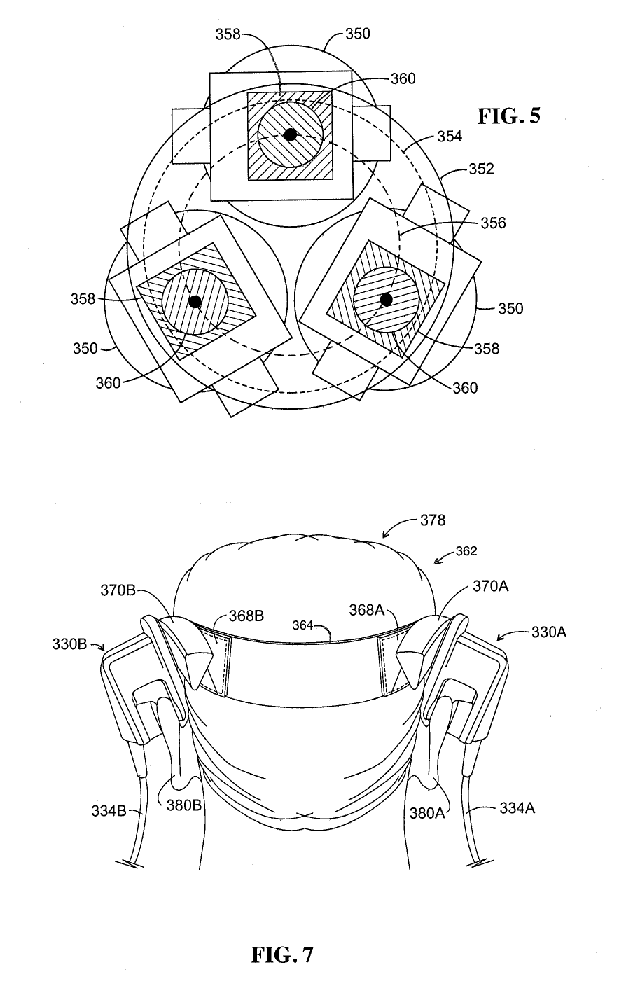

Referring to FIG. 5, an alignment diagram shows the relative positioning of the components of the fluorescence sensor array employed with devices as at array fixture 330 (see FIG. 4). In the figure, the physical diameter of the laser emitter diodes is represented at 350. These devices are identified as Sanyo Laser Diodes, catalog number DL-7140-201S, and have a 0.220-inch diameter. Circle 354 represents the outer diameter of an Andover Corporation (Salem, N.H.) interference filter. (Interference filters of similar configuration are available from Osram Opto Semiconductors of Sunnyvale, Calif.) Circle 356 represents the centerline of the laser diodes and photo diodes. Square 358 represents the active area of the photodetectors marketed by Andover Corporation (Salem, N.H.) and circles 360 represent the elliptical cross-section of the laser beams.

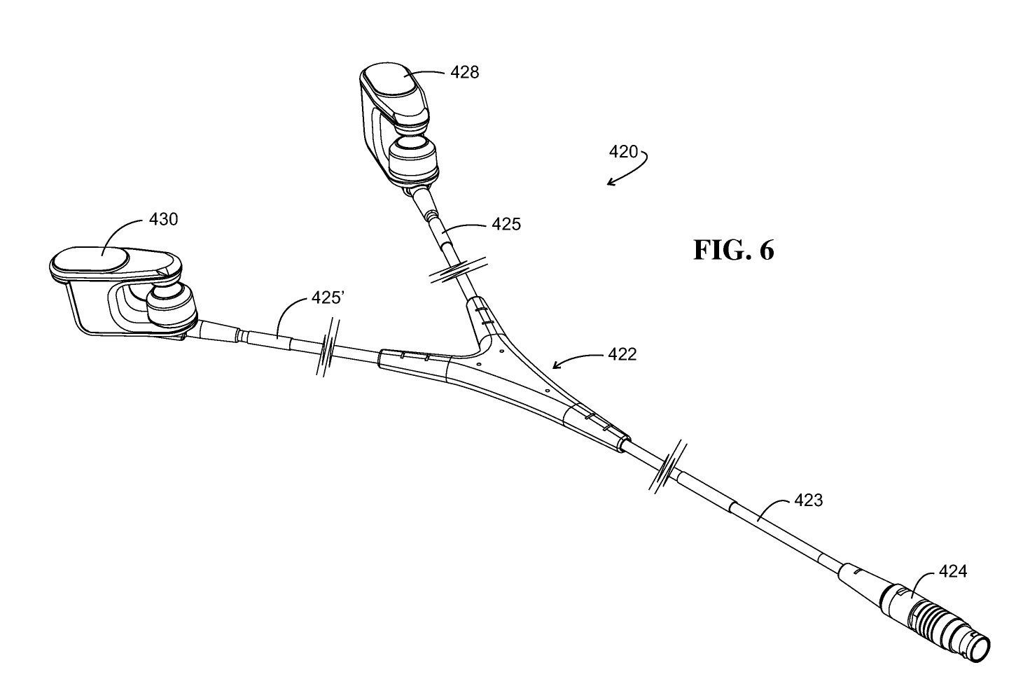

In a preferred embodiment, the sensor arrays are utilized at paired locations on the patient body, e.g., preferably on both ears, fingers of both hands, or both great toes. In order to connect the sensor array apparatus to the monitor controller, a reusable connection cable 420 is provided as seen in FIG. 6. As shown in FIG. 6, communication connector hub 422, a left connecter cable 425, extending from device 428, receives an input as well as provides outputs and is coupled to device 428. In similar fashion, a right connector cable 425′ couples to device 430. Finally, a connector for cable 423 is directed to a monitor controller connector as shown at 424.

FIG. 7 shows a headband system for optimally positioning the sensor array systems on the head and about the human ear. Headband system 362 is positioned on the head of patient 378, with the headband support being comprised of band 364, magnetic adaptor wedges 370A-B and ferrous plates 368A-B. The easily repositioned magnetic wedges effectively couple sensor arrays 330A-B about ears 380A-B of patient 378, respectively, with connector cables 334A-B being in electrical connection with the monitor controller. Scaphoid fossa of ears 380A and 380B slide into the throat of arrays 330A and 330B, and are thus positioned via connector wedges 370 in order to be accurately placed on the scaphoid fossa and avoid impingement on the vasculature of the ear. Sensor arrays can be positioned at other locations on the patient body with adapted support systems.

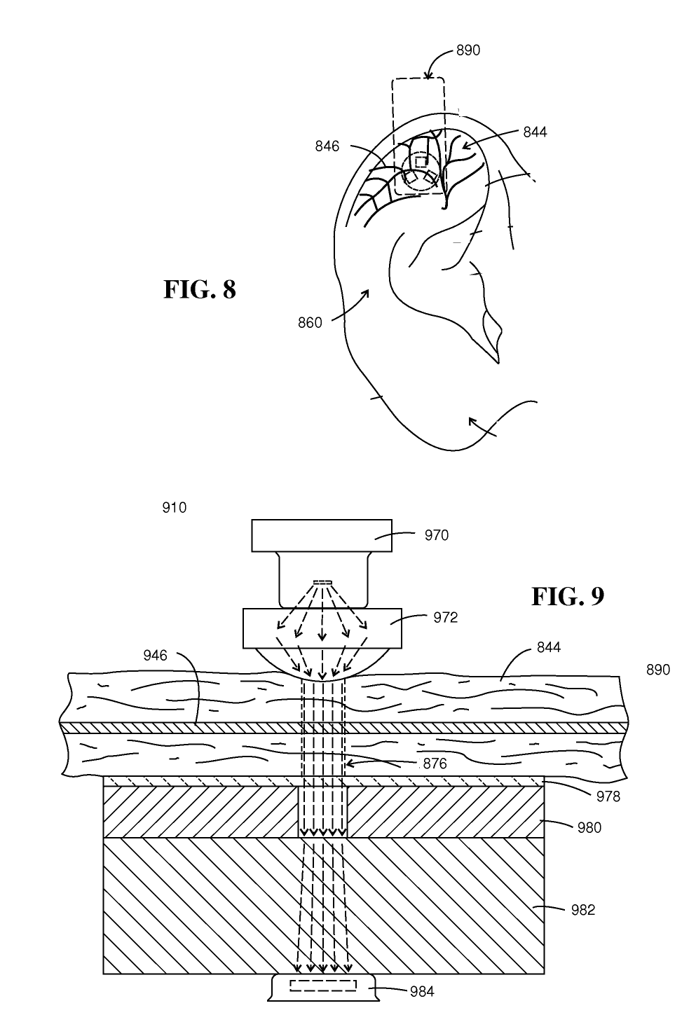

As shown in conjunction with FIG. 8, a portion of tissue at 890 with circulating blood within a blood vessel at 846 can be assayed using a sensor array as disclosed herein. A transmission mode of sensing is shown associated with that part of the ear. The scaphoid fossa 844 of the pinna 860 is vascularized in conjunction with blood vessels 846. As shown in greater detail in FIG. 9, the tissue region 890 is assayed by the sensor array, including a transmission mode sensor 910. The components of the transmission mode sensor can include a photo emitter diode 970. Photo emitter diode 970 may be a type DPW34BS, marketed by OSRAM. (see also, e.g., Sanyo or ADL diode lasers available through DigiKey, Thief River Falls, Minn.) the output of which is associated with an aspheric collimating lens 972. Laser light as represented at 876 is directed into the scaphoid fossa 844 to impinge upon a blood vessel 846. Laser light and fluorescence-generated photons then occur as represented in general at 876, passing a transparent window 978, the bore of an opaque collimator 980, and interference filter 982. Filter 982 passes essentially only the photons resulting from fluorescence to impinge upon a photodetector 984.

Thus returning briefly to FIG. 1 the system is utilized with a method of using a catheter in connection with an injection system to inject an analyte indicator into the patient (generally at 175). Sensor arrays having at least two emitter detector pairs are positioned on the patient, at a vascularized region, e.g. the ears. After the analyte indicator is injected, the monitor controller queries the sensor arrays to detect the presence and disappearance of the analyte indicator in the blood stream of the patient. During the test, and at the conclusion of a testing period, the monitor controller provides a report of the rate of disappearance or modification of the analyte. With respect to the liver, the disappearance of ICG fluorescence from the blood is used to indicate the capacity of the liver to process blood-borne compounds.

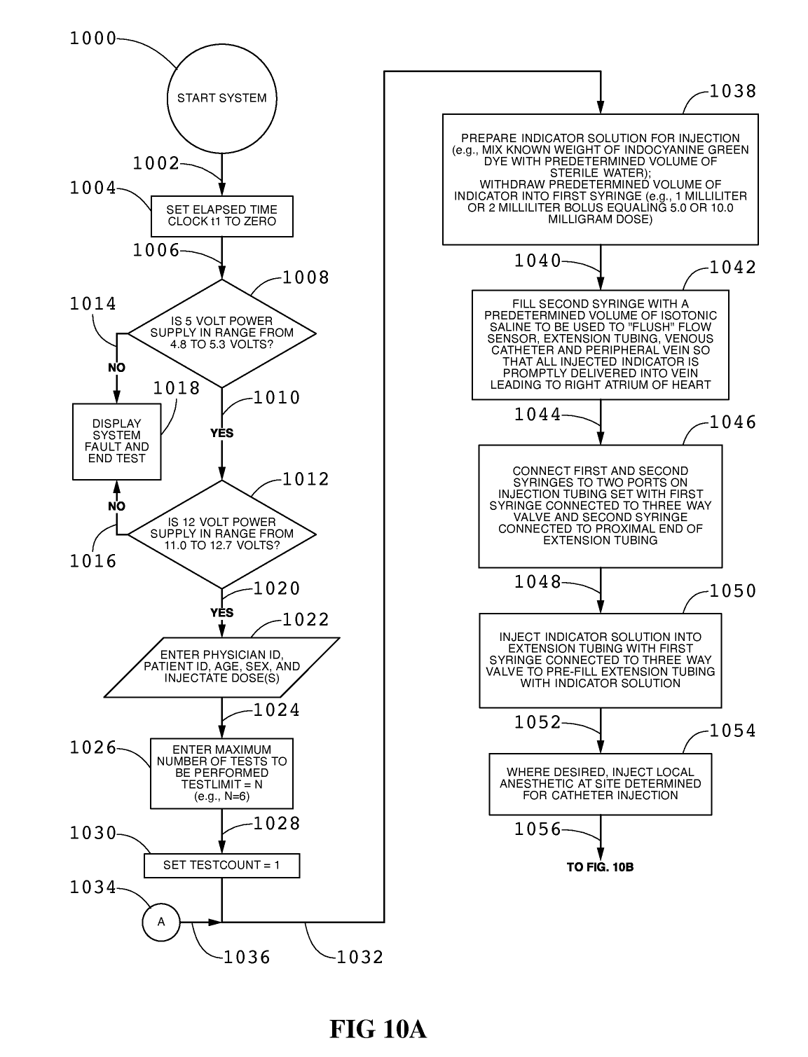

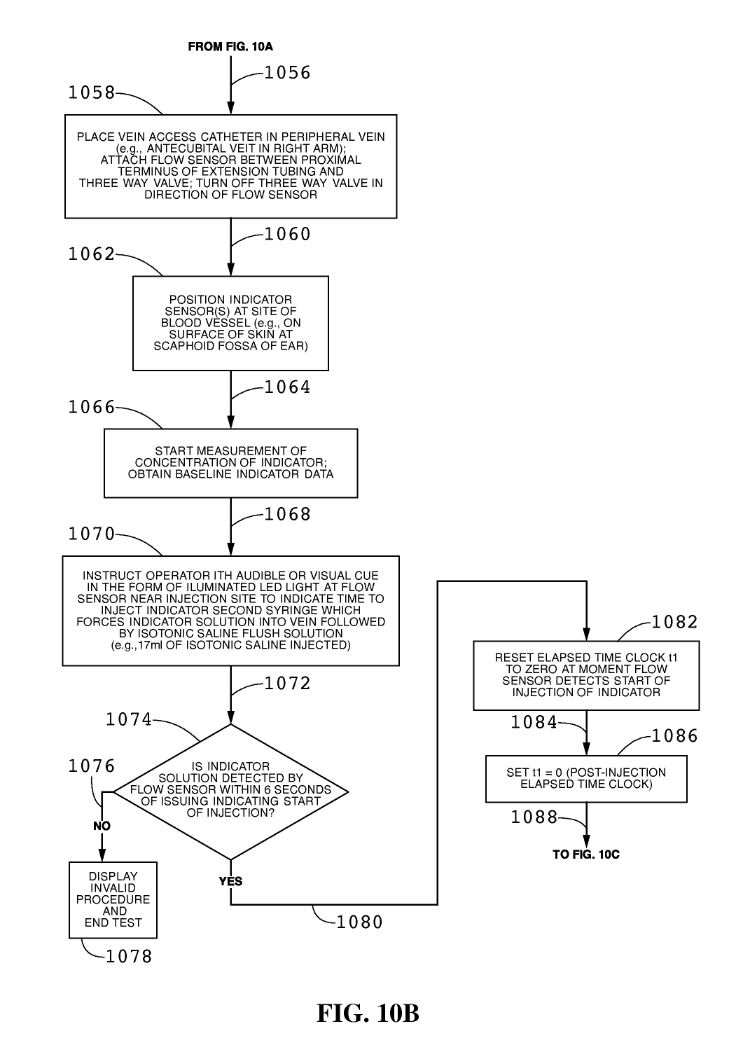

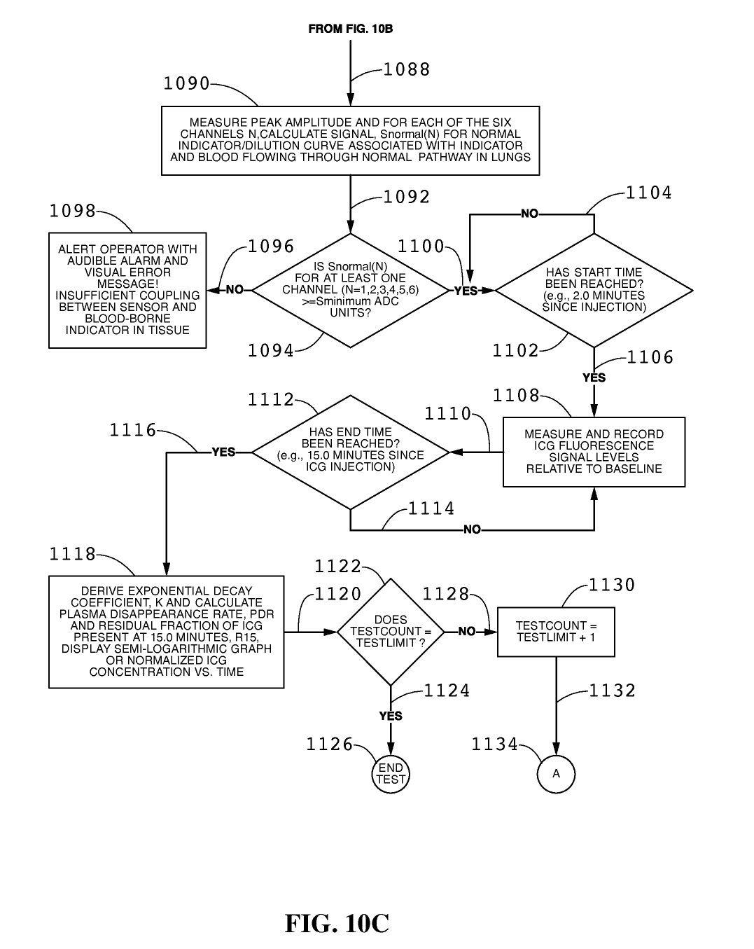

A general flow chart of the operation of the system is described in FIG. 10A-C. The figures thus combined as labeled thereon to provide a flow chart describing the system and method as used to assay organ function. Using the system for monitoring liver function can utilize the following protocol without extensive experimentation. Beginning as represented by symbol 1000 and continuing as represented by arrow 1002 to block 1004, the controller carries out system initialization with default parameters. The elapsed time clock t₁ is set to zero. Next, as represented at arrow 1006 and block 1008, the 5-volt power supply output voltage is measured, and if the measured output falls within the 4.8- to 5.3-volt range, arrow 1010 is followed to block 1012. At block 1012, the 12-volt power supply output voltage is measured and must be within the 11.0- to 12.7-volt range to continue via arrow 1020. If the measured output voltage of either the 5-volt power supply or the 12-volt power supply do not fall within the respective desired ranges, as at 1014 and 1016, respectively, then at block 1018, a system fault is displayed and the test ends.

If the voltage output levels are within the acceptable ranges, arrow 1020 is followed to block 1022, where the physician identification number, the patient identification number, age, sex and intended injectate dose(s) are entered via the monitor touchscreen, or associated keyboard. Following arrow 1024 to block 1026, the maximum number of tests, i.e. dye injections is entered. This number, N, is dependent on the particular analyte being utilized, and for ICG, the maximum number of tests is calculated by determining the maximum daily dose (typically as mg/kg body weight) for the patient, and dividing the maximum daily dose by the injectate dose, i.e. 5 or 10 mg), and then rounding the result to the next lowest whole number.

Next, as represented at arrow 1028 to block 1030 the test count variable is set to testcount=1 and, as represented at arrow 1032 to block 1038, where the injectate (indicator solution for injection) is prepared, for example by mixing a known weight of indocyanine green dye with a predetermined volume of sterile water. A predetermined volume of that mixed indicator is withdrawn into a first syringe. That syringe is shown as 292 in FIG. 2. Note that once prepared, the ICG stock solution has a relatively short acceptable shelf life. The program continues as represented at arrow 1040 to block 1042, where block 1042 provides for filling a second syringe with a predetermined volume of isotonic saline. That isotonic saline is used to “flush” the flow sensor, extension tubing, catheter, peripheral vein, and the like, so that all of the injected indicator is promptly delivered into the vein leading to the blood stream of the patient. As represented at arrow 1044 to block 1046, the first syringe is connected to a three-way valve and the second syringe is connected to the proximal end of the extension tubing, which is in turn connected to a second port on the three-way valve. The three-way valve setup has been described in more detail in connection with FIG. 2. As represented at arrow 1048 to block 1050, the indicator solution from the first syringe is injected into the extension tubing that is in turn connected to the three-way valve, in order to pre-fill the extension tubing with indicator solution. From block 1050 the program continues as represented at arrow 1052 to block 1054, the latter block describing what has been found to be beneficial under certain conditions, in that a local anesthetic may be injected at the site of intended catheter injection. The program continues as represented at arrow 1056, which reappears in FIG. 10B leading to block 1058.

Block 1058 of FIG. 10B provides for placing the vein access catheter in a peripheral vein and preferably in the antecubital vein of one of the arms. The flow sensor is also attached at block 1058 between the proximal terminus of the extension tubing and the three-way valve. The three-way valve is turned off in the direction of the flow sensor. The fluorescent sensing indicators are then positioned at a blood vessel site—the scaphoid fossa of the ears of the patient, for example—as represented at arrow 1060 to block 1062. From block 1062, arrow 1064 leads to the “Test Ready” indication from the monitor at block 1066 determining whether or not the operator is ready to begin the test, and which looks to obtaining base line data.

Arrow 1068 extends to block 1070, which will prompt the operator to inject indicator. In the event that the operator is not ready, the system waits for an operator ready cue or prompt. Then at block 1170 the waits for a positive response to the query posed as to whether the time for instructing injection is present. When the time to inject is present, the practitioner is instructed, first to be ready, immediately followed by instructions to commence the injection of the first syringe, which forces the indicator solution into the vein, followed by the second syringe isotonic saline flush solution. The practitioner may be provided with a visual cue via, for example, an illuminated LED light affixed on or near the flow sensor, so that the cue may be conveyed without difficulty. The flow sensor will detect the flow of indicator, as represented by arrow 1072 to block 1074. The flow sensor will make such a detection within a predetermined time after the injection cue is made to the practitioner at block 1070. For example, at block 1074, the flow sensor attempts to detect the presence of indicator solution for a six second period following its issuance of the cue to indicate the start of the injection. If no detection is made within this time, as at arrow 1076 to block 1078, the procedure is deemed invalid and the test is ended. When such a flow is detected, as represented by arrow 1080 to block 1082, time clock t₁ is set to zero at the moment the flow sensor detects the start of the injection of indicator. At block 1086 t₁, post-injection elapsed time clock is set to t₁=0. Following arrow 1084 to block 1086, time t₁ then begins counting up, to be used to determine first query time t₂, and end query time t₃.

Arrow 1088 reappears in FIG. 10C extending to block 1090. (Although the sensor arrays may optionally be activated upon injection of the analyte indicator, typically t₂, first query time will be set for a time that is equivalent to about 2 to 3 minutes.) Block 1090 measures the peak amplitude, and for each of the channels N, calculates the peak amplitude signal, S_{NORMAL}(N) associated with analyte indicator and blood flowing through a normal pathway that passes through the liver for removal. Where that signal is greater than the minimum signal, then as represented at arrow 1092 and block 1094, the peak amplitude signal for each channel is measured.

Then, as represented at arrow 1092 to block 1094, a query is made as to whether the measured signal for at least one channel is equal to or greater than a minimum designated signal. Where it is not, then as represented at arrow 1096 to block 1098, the practitioner is alerted with an audible/visual error message that there is insufficient coupling between the sensor and blood-borne indicator in the tissue.

When the S(N) for at least one of the channels is greater than the minimum value, next, as represented at arrow 1100 to block 1102, an inquiry is made to whether the delay flag is now zero, i.e. whether the first query time t₂ has been reached. Where t₁<t₂, the query is repeated. When t₁>t₂, the system invokes the actions of block 1108, querying those sensor array channels, N for which S(N) is greater than the minimum value and measuring and recording analyte fluorescence for those channels. If the delay flag=0, then the liver function metrics are calculated and displayed and the semi-logarithmic graph is optionally displayed. For monitoring liver activity, the calculated best fit of the log concentration versus time decay line may be displayed as data is collected.

The measuring and recording function continues until the end time t₃ has been reached. Thus, following arrow 1110 to block 1112, the end time query is posed as to whether t₁>t₃. When it is not, then as represented at arrow 1114 back to block 1108, measuring continues. When the query at block 1112 is answered in the affirmative, following arrow 1116 to block 1118, active measurements are discontinued, and liver metrics are determined and optionally displayed on the monitor.

During or after completion of the calculation steps in block 1118, following arrow 1120 to block 1122, a determination is made as to whether the number of tests performed is equal to the preset test limit. If the test count equals the test limit, arrow 1124 is followed to symbol 1126, and the test is ended. The monitor controller will terminate the access key to the flow sensor apparatus, preventing reuse or an excess number of re-tests.

When the test limit has not been reached, as at arrow 1128, the test count is changed to be test count=J+1 at block 1130. The program then continues as represented at arrow 1132 to Node A 1134 in preparation for a subsequent liver function test, if desired (e.g., the liver function test may be repeated two or more times after a time interval of 60 minutes after the start of the preceding test). Node A reappears in FIG. 10A, where arrow 1036 leads to the prompts for a repeat test.

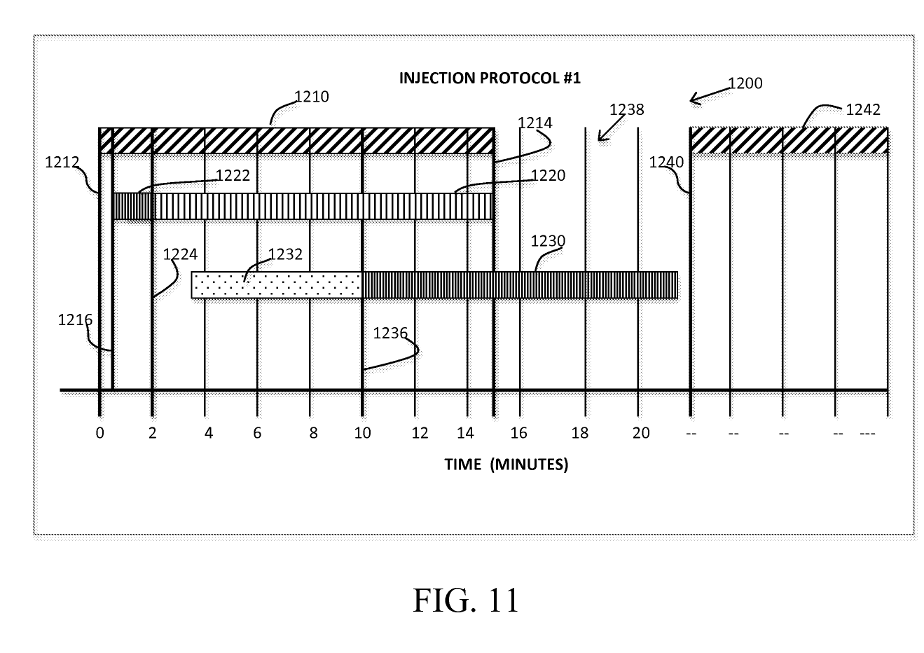

The course of the procedures is summarized in a Chart 1200 shown in FIG. 11 as what is referred to as Protocol 1 by way of example. In the figure, a fifteen minute testing procedure is shown as bar 1210. Vertical line 1212 shows the initiation of a first test by injection of the analyte indicator into the bloodstream. The first test period extends to vertical line 1214. Beginning at vertical line 1216, the system is first able to detect indicator analyte signal above background Bar 1220 shows the period during which the sensor array is typically configured to collect data for determining liver metrics, said period typically extending from about 30 seconds to about 15 minutes following injection. Initial period 1222 of bar 1220, extending from about 30 seconds to about 2 minutes post injection represents the period of mixing of analyte indicator in the blood of the patient. Bar 1230 represents the period during which the monitor/controller may display or report liver activity metrics, with an initial period 1232 which may begin soon after about vertical line 1224, and extending to vertical line 1236. After vertical line 1236, the liver activity metrics are predicted to have sufficient reliability for clinical analysis. It should be recognized that depending on the quality of data obtained by the system, the initial period may be much shorter. The period during which results are displayed or reported, as shown by bar 1230 may extend beyond vertical line 1214, so long that a renewed testing procedure is not undertaken, and the monitor/controller remains available. A dwell period at 1238 of variable duration, lasting about 30 to 45 minutes is typically implemented before another testing procedure can be initiated with the same patient. The figure represents a new procedure beginning with vertical line 1240, and continuing as shown by bar 1242. (The details of the procedure depicted by bar 1242 are not shown in FIG. 11.) Other protocols may be implemented in keeping with the system disclosed, and compatible with a particular analyte/organ assay being contemplated.

In a preferred embodiment, the liver function metrics just described are calculated as described in the following steps. The first step, after digital filtering of the raw data for each of the six channels of ICG concentration vs. time data, is to use an off-the-shelf exponential curve fit software algorithm to determine the exponential coefficient of the equation listed below:

C[t2]=C[t1]*Exp{−K*t2} (Equation 1)

Where:

C[t2]=concentration of ICG relative to Baseline ICG concentration at elapsed time, t2 with time, t2 expressed in units of minutes and concentration expressed in units of millivolts

C[t1]=concentration of ICG relative to Baseline ICG concentration at elapsed time, t1 with time, t1 expressed in units of minutes and concentration expressed in units of millivolts; t1 would be starting time for exponential curve fitting (e.g., 2.00 minutes)

K=ICG exponential decay clearance coefficient expressed in units of reciprocal minutes

The ICG Exponential Decay coefficient, K is computed for each of the six channels for a range of starting and ending times. The matrix will include 2.0 and 3.0 minutes as the starting time and ending time of 10.0 to 20.0 minutes. The “goodness of fit” parameter computed using the selected exponential curve-fitting algorithm may be used to select the best channel of the six available data channels.

In this curve fitting step, the curve fitting involves dividing C[t2]/C[t1] for a range of C[t2] and a constant C[t1] value (i.e., the relative ICG concentration value at 2.00 minutes). Once the K value is derived, then solve Equation 1 for elapsed time, t2=0.0 minutes and setting C[t1]=100%. Re-plot semi-logarithmic graph (e.g., originally plotted using relative ratios of C[t2]/C[t1] for t1=2.00 minutes and t2 ranging from 2.5 to 10.0 minutes) with the maximum ordinate value set equal to 100%.

Once the exponential decay coefficient, K (in units of reciprocal minutes) is derived as described above, the Plasma Disappearance Rate, PDR (in units of % per minute) is determined by multiplying the exponential decay coefficient by 100.

PDR(%/minute)=K*100 (Equation 2)

The residual fractional ICG concentration at 15 minutes after ICG bolus injection, R15 is given by the following equation:

R15(%)=100%*Exp(−K*15.0 minutes) (Equation 3)

An advantage of the enhanced sensitivity of the disclosed system is that it allows regular testing of liver activity, without exceeding the daily allowable dose of liver analyte indicator. Present systems may require using the full available dose for a single test, while the present system may allow ten or more tests over a given 24 hour period. When utilizing a rapidly metabolized indicator delivered at low concentrations, frequent determinations of liver function metrics is practical (e.g., at an interval of 30 to 90 minutes between each test). It is a substantial advantage of the present system to allow near real-time monitoring of the activity of organs. By displaying the present activity of an organ such as the liver or kidney, a drop in organ activity due to insult, trauma, or disease can be identified prior to the patient entering a critical phase when liver or kidney failure is already advanced.

The present system is applicable to non-human patients, as well as human patients. In general the system is operable with a variety of mammalian patients, including working animals, such as dog and horse, and laboratory animals such as pig, sheep, and rabbit. In particular, certain very valuable animals, such as pets, companion animals, race horses, and show horses may at times be afflicted with disease. As such, the disclosed monitoring system can be readily utilized in conjunction with essentially any large mammal of interest, and adapted to use with small laboratory animals such as rat and hamster.

As is clear from the forgoing disclosure, identification of an efficacious analyte is of particular importance, and specifically looking to the liver activity indicator, a circulating tracking reagent is called for. Studies at the outset of the research leading to the present invention indicated that a preferred embodiment was to employ fluorescing dyes, certain of which had been approved for use in humans. Two such exemplary dyes include fluorescein and indocyanine green dye (ICG). As disclosed above, ICG as an analyte indicator of liver activity is a preferred embodiment. By no means, however, is the present disclosure restricted to ICG as an analyte indicator.

A number of additional analyte indicator reagents are available for use with the system at hand including such indicators as follows: U.S. Pat. No. 3,412,728 describes the method and apparatus for monitoring blood pressure, utilizing an ear oximeter clamped to the ear to measure blood oxygen saturation using photocells which respond to red and infrared light; U.S. Pat. No. 3,628,525 describes an apparatus for transmitting light through body tissue for purposes of measuring blood oxygen level; U.S. Pat. No. 4,006,015 describes a method and apparatus for measuring oxygen saturation by transmission of light through tissue of the ear or forehead; and U.S. Pat. No. 4,417,588 describes a method and apparatus for measuring cardiac output using injection of indicator at a known volume and temperature and monitoring temperature of blood downstream. This and several similar systems in the art suffer from an inability to effectively quantify the magnitude, i.e., functional conductance of shunts, as opposed to the presently disclosed embodiments.

As disclosed herein, preferred indicator analytes are capable of circulating in the blood or perfusing other tissues and/or fluids, and the indicator analyte is associated with a fluorescent moiety that can be excited to emit fluorescence through irradiation by excitation photons in wavelength range known to induce fluorescence in said fluorescent moiety. In certain cases, the metabolism of the patient body may activate the moiety, or otherwise result in the moiety losing its fluorescent capacity. The correlation of the action of an organ of interest may be established with the relative availability of the fluorescent moiety or fluorescence capacity therein.

A number of patents describe potential reagent systems that if adapted could be utilized with the present system method and apparatus. U.S. Pat. No. 4,804,623 describes a spectral photometric method used for quantitatively determining concentration of a dilute component in an environment (e.g., blood) containing the dilute component where the dilute component is selected from a group including corporeal tissue, tissue components, enzymes, metabolites, substrates, waste products, poisons, glucose, hemoglobin, oxy-hemoglobin, and cytochrome. The corporeal environment described includes the head, fingers, hands, toes, feet and ear lobes. Electromagnetic radiation is utilized including infrared radiation have a wavelength in the range of 700-1400 nanometers. U.S. Pat. No. 6,526,309 describes an optical method and system for transcranial in vivo examination of brain tissue (e.g., for purposes of detecting bleeding in the brain and changes in intracranial pressure), including the use of a contrast agent to create image data of the examined brain tissue.

Looking to the indocyanine green dye (ICG), excitation curves have been illustrated as having a peak excitation wavelength at about 785 nanometers. Correspondingly, for the fluorescent emission of the two fluorescent dyes, a peak wavelength of fluorescing photons resides at about 830 nanometers.

The transmission mode of sensing as described in connection with FIG. 9 finds advantageous application at regions of the body in which surface tissues are relatively thin. The transmission mode of sensing is preferably applied to locations of the patient body wherein the vasculature, including both the venous vasculature, and the arterial vasculature, is arranged such that the transmissive sensors can be placed opposite the photodetectors in a noninvasive manner. Preferred locations on the human body include the scaphoid fossa of the ear, the finger of each hand, the hand, including the web of skin between the thumb and forefinger, the neck, including distendable skin about the neck, the leg, and the arm, including distendable skin of the arm proximal to the shoulder. Non-human patients, such as dog, pig or horse, also provide ready locations for sensors on the ear, in addition to other vascularized extremities. A preferred embodiment of the system is to place sensor arrays at symmetrically paired locations distal to the heart, such as at is both ears, both hands, paired locations on the neck, the leg, and the arm. A particularly preferred embodiment is placement of sensor arrays on both scaphoid fossa of the ears of the human patient and identified generally at 844 of FIG. 8. The system is readily adaptable and configurable for use with a subject that is a laboratory animal, a zoological specimen, a cat, a dog, an elephant, an ape, a horse, or a human, for instance.

The system outlined in FIG. 1, and the disclosure in connection therewith, is useful for testing a variety of parameters useful for optimizing the system and apparatus. Although ICG is the presently preferred indicator dye, other dyes may be even better adapted, and proof of concept of the system, using tissue phantoms, actual tissue and other factors can be readily screened as described in the examples that follow. Moreover, improvements to the detection system itself are amenable to bench top testing for system optimization.

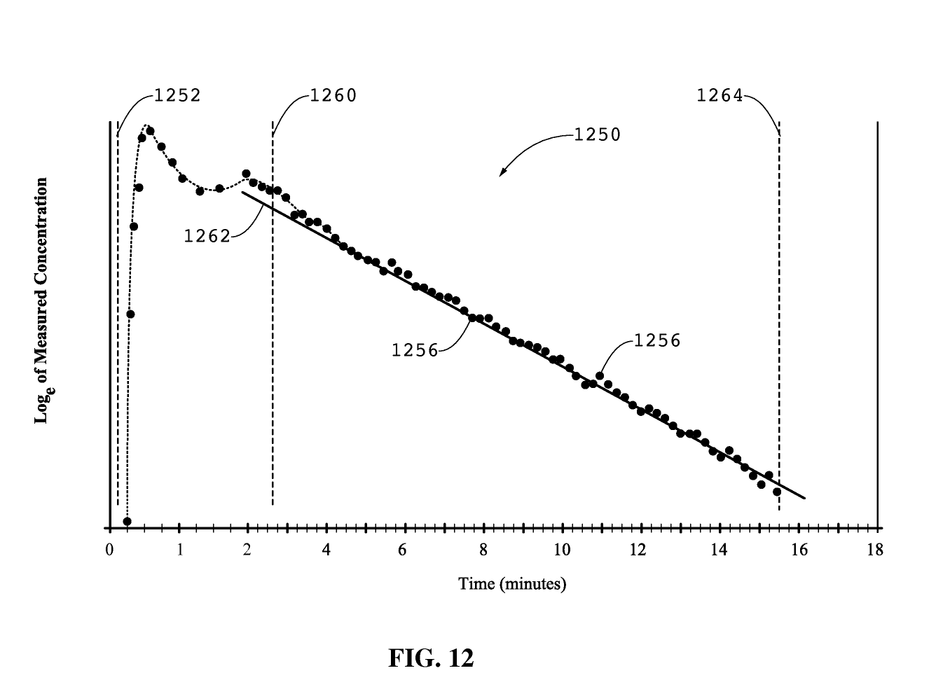

Referring to FIG. 12, a hypothetical graph 1250 is shown displaying hypothetical measured analyte concentrations following an analyte injection into a patient. Vertical line t₀ at 1252 represents the detection of the injection of analyte bolus by the flow sensor. Soon after, approximately 30 seconds, for instance, the system detects the presence of fluorescing analyte. As shown in the example graph, the system takes repeated readings, as at 1256, to determine a measured analyte concentration, the frequency of said sampling being, for example, about 5 times per minute, in a preferred embodiment 16 times per second for the first two minutes and then 6 times per minute, thereafter. Time, t1, begins counting up upon first detection of analyte. In the initial phase corresponding to the period during which the ICG is nonuniformly distributed within the circulating blood stream, the measured concentration will rapidly peak, and then once the analyte has been uniformly distributed throughout the blood and is being extracted from the blood stream by only the liver, the relative concentration will decrease according to the liver extraction capacity of the patient. As the analyte is extracted, the analyte concentration is expected to follow an exponential decay curve. By way of example, the first query time t₂, the starting time for the exponential decay curve, is shown as vertical line 1260 specified as about 2.0 minutes. Best fit line 1262 is preferably fit to data from the period beginning with first query time t₂ and continuing until the signal is diminished or the test is ended, at end query time t₃. End query time t₃ is represented by vertical line 1264 on graph 1250. The ending time for this example liver function test is approximately 15.0 minutes. The rate at which the concentration decays is representative of the ability of the organ (liver) to process the analyte indicator (ICG). A normal liver is represented by decay curve 1262, while a diseased or otherwise compromised liver would be expected to display a line with a reduced slope, or that did not conform to a straight line on a semi-logarithmic graph throughout the test period.

In general, the controller circuitry used with the system will compute the exponential decay shown as solid line region 1262. As stated above as related to Equations 1, 2 and 3, the measured ICG fluorescence signal levels relative to baseline (following digital filtering to remove heart beat artifact and other sources of higher frequency noise) are recorded in this example over the period from about 2 to 15 minutes. Decay curve 1262, which is preferably a straight line when plotted on a semi-logarithmic graph can be displayed on the system monitor, with or without the representation of data points, as shown in FIG. 12. Thus, FIG. 12 is representative of a hypothetical monitor display or printed report.

The recorded values are then analyzed using an exponential curve fitting algorithm to derive the exponential decay coefficient, K that provides the best fit (i.e., least error) to the measured data. Once the exponential decay coefficient, K is derived then the Plasma Disappearance Rate, PDR value can be derived as specified in Equation 2 above. The residual ICG relative concentration level at 15 minutes, R15 can be calculated as specified in Equation 3 above and/or can be derived based on the actual measured relative ICG signal level at an elapsed time of 15.0 minutes. A normal liver is generally characterized by a Plasma Disappearance Rate 15% per minute or greater. A Plasma Disappearance Rate 4% to 10% per minute is generally associated with a diseased or otherwise compromised liver.

It is also contemplated that rather than having discreet injections of an analyte, a continuous delivery of analyte could be produced by use of an IV drip, or measured perfusion pump to deliver the analyte. In such a situation, the monitor could display a second order calculation showing the change in organ activity over time. Thus, a curve 1460 shows the instantaneous liver activity, while a curve from continuous perfusion could display a measure of sustained the liver processing capacity.

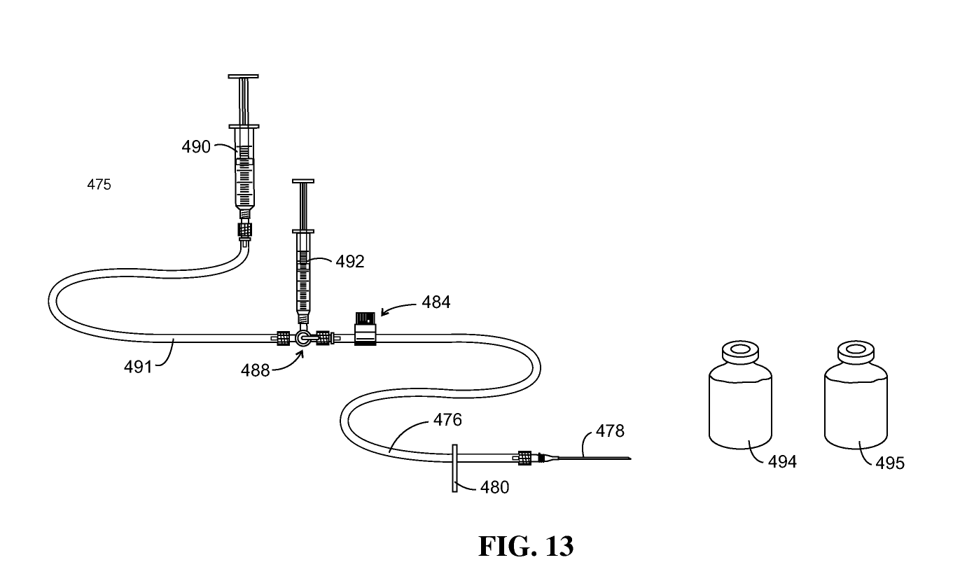

A further embodiment of the system is a kit supplying consumable materials necessary for quantifying a circulatory anomaly. FIG. 13 shows the contents of one version of a kit for providing the necessary consumable materials and providing for safety checks for utilizing the apparatus. Indicator delivery tubing system, shown generally at 475, provides a single use apparatus for performing the injection procedure. Delivery tube 476 terminates in catheter connection 478, or as a needle suitable for intravenous injection. Flow sensor 484 connects to the system, providing for logging the initiation of injections, and is clamped about tube 476. As described, a single use flow sensor is preferred, providing for a safety factor that apparatus such as tubing set 475 is not reused to the potential detriment of patients. Clip 480 allows secure attachment of the delivery tubing to the apparatus or patient. Three-way valve cock 488 allows the practitioner to load tube 476 from syringe 492, and then switch to a connection with tube 491, which allows flushing of the contents of tube 476 with the contents of syringe 490. Vial 494 comprises one or more doses of indicator dye reagent as a shelf stable material. Vial 495 is a saline diluent for preparing the dose of indicator dye reagent for injection into a patient; a syringe and needle apparatus for mixing the dose of indicator dye reagent and the diluent. The syringe and needle provided are suitable for injecting the indicator dye dose into the system injection port, and will typically be supplied as a first and second syringe suitable to introduce the indicator dye reagent and saline bolus into the patient. Finally, a saline solution, for instance, is provided to supply a dose of nonreactive blood-compatible clearing reagent for completing the injection and pushing the indicator dye dose into the bloodstream of the patient. Finally, in order to ensure patient safety, all the contents of the kit can be packaged in a single sterile package, such as a sealed plastic tray containing the kit contents in a sterile condition until opened. Sterility can be accomplished, for example, by ethylene oxide gas sterilization (excluding the ICG which is received pre-sterilized from the manufacturer (e.g., Pulsion Medical or Akorn, Inc.). Typically, the kit will be supplied in a sealed tray containing the kit contents maintained in a sterile condition until opened.

### EXAMPLES

The following examples are provided to more fully explain the system and apparatus. However, they should not be viewed as limiting.

### Example 1

### Liver Activity Assay Trials

Objectives of prospective indicator dosing trials and comparative analysis tests include optimization of the injection protocol to further increase the system sensitivity for monitoring liver function. Another objective is to determine test procedure parameters in preparation for subsequent trials. Further objectives include providing additional data for developing the disclosed method for the calculation and display of the functional flow conductance of a patient's liver. The following protocol demonstrates a testing procedure for determining the ability of different analyte indicators to assay organ function. In particular, the following protocol is designed for demonstrating efficacy of liver targeted analytes. Similar protocols can be readily implemented for demonstrating the efficacy of analytes suitable for assaying other organs. Undue experimentation is not necessary to demonstrate the efficacy of any analyte for use with the minimally invasive organ assay systems disclosed herein.

A kit, similar to that disclosed in relation to FIG. 13, is to be provided in a single-use procedure tray. The kit contents included Indocyanine Green (ICG) dye (Pulsion Medical Systems AG, Munich) as a vial containing 25 mg of ICG powder. A second vial provides solvent for preparation of a solution of ICG dye solution at the desired concentrations. The kit also would contain a single-use, sterile catheter set with associated flow sensor.

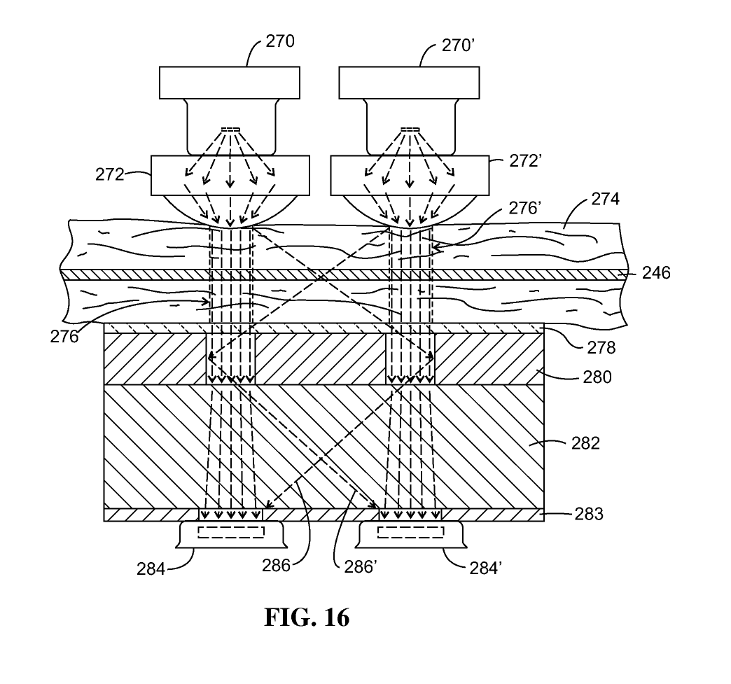

Two reusable Fluorescence Sensor Array units, of the type disclosed in FIG. 4 or 16 are to be connected to the Controller/Monitor via a cable (as shown in FIG. 6), providing for the measurement of fluorescence-based ICG concentration level measurements at six sensor locations. Each of the Fluorescence Sensor Array (FSA) units are comprised of three independent transmissive sensors, and are positioned at the scaphoid fossa of each ear of the patient, as illustrated in FIG. 7. The power level and the duration of the laser pulses are selected to meet laser safety requirements, with the maximum power delivered within the laser beam being less than 0.28 watts/sq. cm. (below the recommended Maximum Permissable Exposure (MPE) of 0.30 watts/sq. cm. specified in Table 7 of ANSI Z136.1-2007). Utilizing the disclosed optical filtering and collimation to block the 785 nm excitation photons, the emitted fluorescence photons are selectively received by a photodetector, digitally processed and recorded by the Controller/Monitor unit as described above. The use of multiple channels (viz. three at each ear) allows for analysis of the positioning of the sensors, and the sufficiency of a three sensor array in providing that at least one sensor (channel) would always be closely positioned relative to an underlying and invisible blood vessel in the Scaphoid Fossa region of the patient's ears, or other vascularized and accessible tissue. Thus, by utilizing a pair of three sensor arrays, the probability of a high sensitivity test result is increased.

A single-use, sterile catheter set is connected to an AngioCath catheter similar to that illustrated in FIGS. 1, and 3. Within 30 minutes of initiating the test, the ICG powder supplied is reconstituted with sterile water, as described in its package insert, to create an ICG dye solution having a concentration of 2.5 mg/ml. This ICG dye solution is either then injected at this concentration of 2.5 mg/ml or further diluted with isotonic saline to yield a concentration of 1.25 mg/ml. The catheter set provides the means to either (a) sequentially inject a bolus of ICG dye followed by an isotonic saline flush or (b) inject using a single syringe of either dilute ICG or a pre-loaded bolus of ICG pushed by a 17 ml volume of isotonic saline.

A 20 gauge Angiocath AutoGuard catheter (Becton, Dickinson and Company, Franklin Lakes, N.J.) is first placed in a vein in the antecubital fossa and is subsequently used in the method for assaying liver function.

The supplied, single-use Catheter Set (see FIG. 13) is next connected to the Angiocath catheter in preparation for the performance of the test. A transcutaneous fluorescence sensor is placed at the scaphoid fossa of both the left and right ears as illustrated in FIG. 7. As shown in FIG. 5, a total of three independent sensor channels are provided on the Fluorescence Sensor Array (FSA) unit placed at the Scaphoid fossa of each ear. The use of multiple sensor channels at each ear greatly increases the probability that at least one channel of one of the two FSA units will be closely aligned with an underlying blood vessel within the scaphoid fossa of one of the ears.

The patient is next instructed by the display on the Monitor/Controller unit to remain still for the next 15 to 20 minutes while the ICG signal levels are continuously measured and recorded. Within about one minute after the end of the test period (nominally two minutes after dye injection), the monitor displays a graph showing the recorded ICG concentration levels from the six fluorescence sensors over the 15-minute period of the test.

### EXAMPLE 2

### Injection/Drug Delivery Recording Device

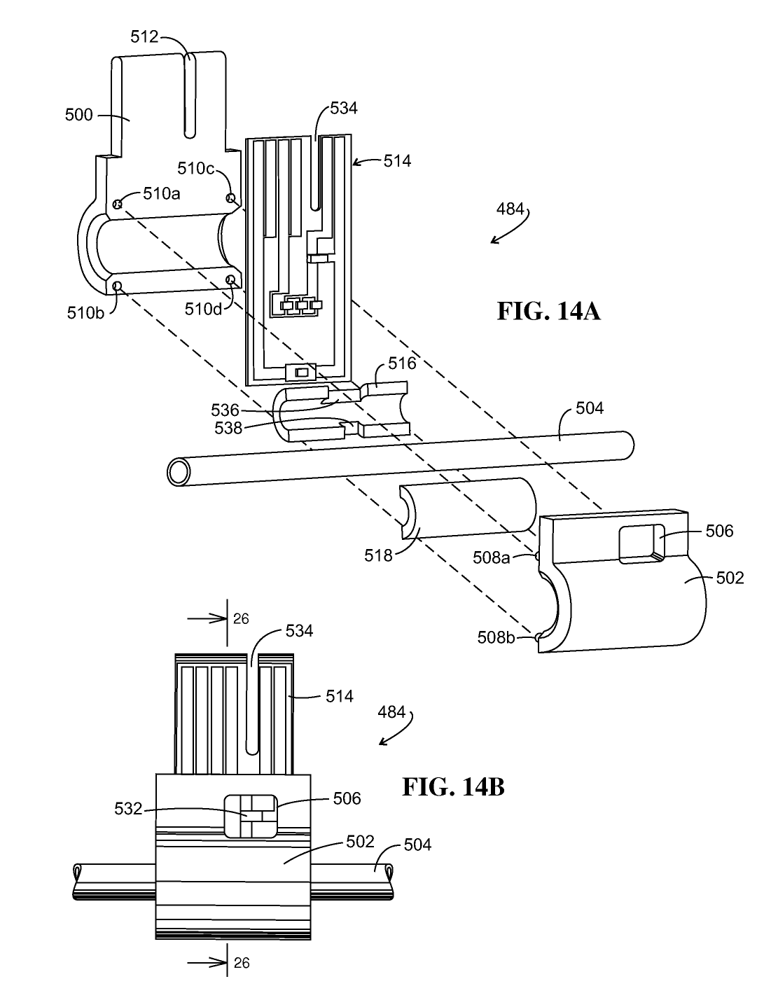

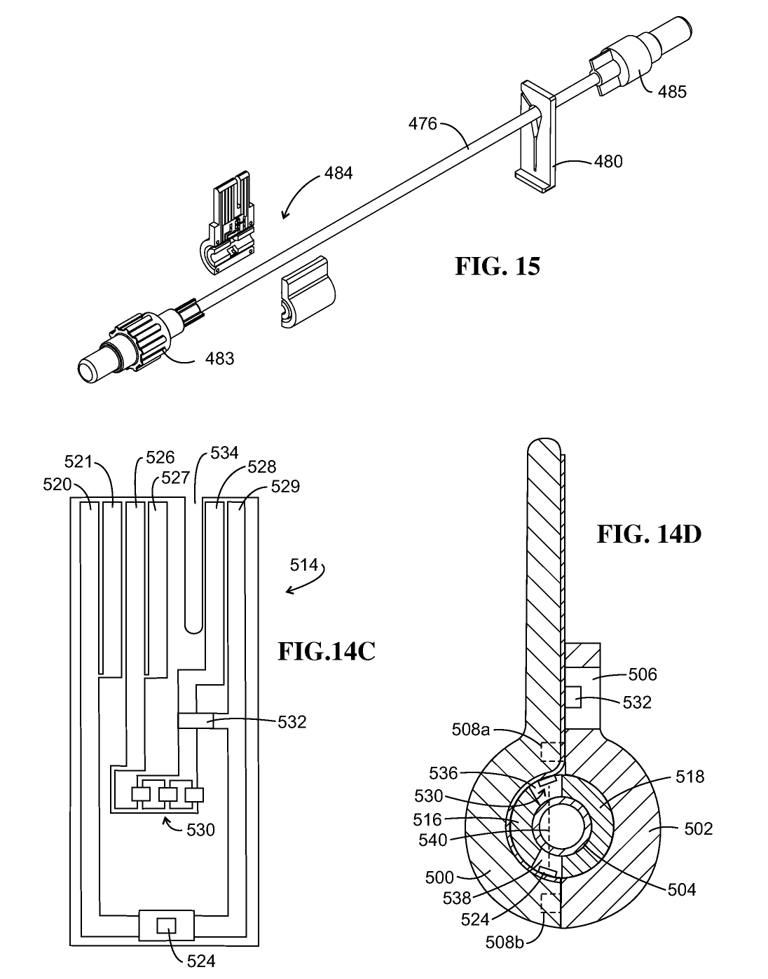

Referring to FIGS. 14 and 15, a dye flow detector 484 is revealed in enhanced detail. FIG. 14A shows two inter-connectable clamp housings 500 and 502 placed on either side of the portion of delivery tubing 504. Additionally, clamp-housing 502 is configured with 4 pins, two of which are seen at 508a and 508b. Two similar pins (not shown) are located on the opposite side of clamp housing 502. These pins are intended to be inserted within holes 510a-510b, within clamp housing 500. Note additionally that clamp housing 500 has a slot 512 formed therein, which provides connector registry. Device 484 performs in conjunction with a flexible circuit shown generally at 514. Flexible circuit 514 is retained in a wrap-around orientation by oppositely disposed support components 516 and 518.

Turning to FIG. 14C, the flexible circuit 514 is represented at a higher level of detail. In that figure, outboard printed circuit leads 520, 521 and 529 extend to a laser 524. Leads 526, 527 and 528 extend to an array of three photodetectors shown generally at 530. A fuse 532 extends between flat leads 528 and 529.

As shown in FIG. 2, the flow sensor connector cable terminates on one end with a flow sensor connector 275, into which a flow sensor is inserted to conduct a test. The connective receptacle includes contacts for blowing the flow sensor fuse to prevent unsafe reuse of the reusable testing kit components. Because repeated tests may be performed on a patient over a period of up to 6 hours, for instance, in an alternative embodiment, the flow sensor may also be configured with a readable serial number or identifier, so that once the particular device ID (i.e., kit contents) is utilized, the flow sensor cable and sensor array components can be reconnected for so long as the kit contents expiration has not been exceeded. Thus, the flow sensor is further embodied as a flow initiation sensor with an initiation sensor, the initiation sensor configured with a circuit that is in communication with the monitor-controller, and responds to a query for determining the number of injections the flow initiation sensor has cued. Once the allowable number or time period has been exceeded the flow sensor can be disabled for repeat use. Note that the shelf stability of ICG is currently approved for 6 hours. Once the first test using an ICG kit sample, has been completed, the monitor/controller can count down the time until the particular kit expires.

The flow sensor as shown can be utilized to monitor and record the injection of analytes for assaying organ function. Thus, a single injection at a given time may be recorded by the monitor controller by way of the flow sensor, in order to initiate measurement with the sensor arrays. Alternatively, if an analyte/indicator dye is being continuously delivered to the patient, for instance through an IV bag, the flow sensor can monitor and allow recording of the amount of indicator delivered and allow the indication of dye delivery to be integrated by the monitor/controller.

The construction of the monitor/controller is shown generally in the applications to which priority is claimed. Schematics for the fluorescence sensor arrays described in connection with FIGS. 4 and 16 herein are also shown in the priority documents. Signal leads provide the communicative pathway between the optional left and right fluorescent sensing arrays and the fluorescent sensing array connector that is coupled to the monitor/controller as described in connection with FIG. 6 (at connection 762). The detection signals are collected at these arrays, 430 and 428, and are transmitted to the monitor/controller for further processing and calculation.

### EXAMPLE 3

### Optimization Parameters for Multi-Emitter/Detector Arrays

Following experimentation, including using animal models, it was determined that sensitivity of the sensor array could be improved by the implementation of multiple emitters and detectors in a sensor array. In order to utilize low concentrations of analyte, such as ICG, sensitivity is highly preferred for assaying organ function. Once a multiple emitter/detector sensor array was implemented, it was recognized that the overall system sensitivity was being hampered by the efficacy of the bandpass filter and collimating plates that were limited by cross talk between related channels in the sensor array. It should also be noted that such cross talk may be even more pronounced when utilizing reflectance mode excitations and detection. The interference filter is necessary in order to reduce incident light arising from the excitation lasers, with the detectors being tuned to detect light emitted as a result of fluorescence. When the interference (i.e. bandpass) filter is ineffective, the excitation light may overwhelm the detection system. As shown by way of example in FIG. 16, an emitter and detector pair analogous to that shown in FIG. 9 is accompanied by another emitter/detector pair, forming a sensor array. (Although 2 emitter/detector pairs are shown, it is recognized that three or more such pairs are preferred.) A portion of the tissue being assayed again is identified at 274 in conjunction with an blood vessel at 246. The laser emitters are represented at 270 and 270′ with their output being directed onto aspheric collimating lenses 272 and 272′. Laser light as represented at 276 and 276′ is directed into the tissue 274 to interact with indicator present in arterial vessel 246. Laser light and fluorescence generated photons then continue, until passing transparent window 278, multiple bores of an opaque collimator 280, and interference filter 282.

Interference filter 282 is designed to pass essentially only the photons resulting from fluorescence to impinge upon photodetectors 284 and 284′. However, when emitted laser light interacts with tissue 274, a portion of such light is scattered, as shown in part by dashed lines 286 and 286′. Such scattered light would be prevented from entering the detector when a single emitter is present, by the first collimating plate 280. When multiple emitters are present, the scattered light may strike the interference filter 282 at an angle less than perpendicular. Since the filter is most efficient when the angle of incidence is 90 degrees, as the angle of incidence is reduced, scattered light (such as excitation laser light) as at 286 and 286′ can pass unimpeded through the filter, and substantially increase the noise detected by the photodetectors 284 and 284′. Recognizing this phenomenon, a preferred embodiment of the array system provides an additional second collimating plate as at 283, thereby maximizing the efficiency of the interference filter, and reducing the light of low angle of incidence that can pass through the interference filter.

As is known, the performance of interference filter 282 is dependent upon the angle of incidence of photons reaching it. Performance degrades as the angle of incidence increases. FIG. 20 demonstrates that use of multiple laser emitters in combination transmitting light through tissue exacerbates the approach of scattered light at a high angle of incidence. The collimating plates as shown in FIG. 16, help to minimize the approach of light at an angle of incidence that can “escape” the bandpass filter. Based on testing of the multi-emitter/detector system, a preferred embodiment has been identified as utilizing a second collimating plate with an aperture of approximately 0.081 inch and a plate thickness of 0.082 inches.

A bench top, ex vivo, or animal model system is useful for testing a variety of parameters useful for optimizing the system and apparatus. Although ICG is the presently preferred indicator dye for assaying liver activity, other dyes may be even better adapted, and proof of concept of the system, using tissue phantoms, actual tissue and other factors can be readily screened with the apparatus shown in the referenced patent applications, including co-pending U.S. patent application Ser. No. 12/754,888, filed Apr. 6, 2010, which is incorporated herein by reference. Additional dyes useful for assaying other organs or organ systems may be tested using the referenced system. Moreover, improvements to the detection system itself are amenable to bench top testing for system optimization.

The present application herewith provides reference to U.S. application for patent Ser. No. 12/418,866, filed Apr. 6, 2009 and entitled “Hemodynamic Detection of Circulatory Anomalies” which, in turn, makes reference to U.S. Provisional application Ser. No. 61/156,723, filed Mar. 2, 2009, and to U.S. Provisional application Ser. No. 61/080,724, filed Jul. 15, 2008, the disclosures of which are incorporated by reference. Also, all citations referred herein are expressly incorporated herein by reference. All terms not specifically defined herein are considered to be defined according to Dorland's Medical Dictionary, and if not defined therein according to Webster's New Twentieth Century Dictionary Unabridged, Second Edition.

Since certain changes may be made in the above-described system, apparatus and method without departing from the scope of the invention herein involved, it is intended that all matter contained in the description thereof or shown in the accompanying drawings shall be interpreted as illustrative and not in a limiting sense. The disclosed invention advances the state of the art and its many advantages include those described and claimed.

We claim:

1. A system for measuring the concentration of a liver activity analyte that is predominantly removed from the circulating blood by the liver and that contains a fluorescent moiety that can be excited to emit fluorescence through irradiation by excitation photons in wavelength range known to induce fluorescence in said fluorescent moiety of a liver activity indicator analyte, comprising
providing an indicator delivery system having an outlet located in a blood vessel of the patient in blood flow communication with the heart;
providing at surface of the skin of a human subject one or more sources of excitation photons in wavelength range known to induce fluorescence in said fluorescent component of liver activity indicator;
providing at the surface of the skin of a human subject and in alignment with a fraction of the emitted fluorescence photons excited by the source of excitation photons one or more detectors to measure the intensity of emitted fluorescence photons from the liver activity indicator; and
providing a monitor/controller responsive to said one or more detectors to record the time-varying relative concentration of the liver activity indicator by recording the time-varying fluorescence signal level.
a sensor comprising a photodiode emitter energizable to generate light at the excitation radiation wavelength and a photodetector which is filtered for response substantially only to the fluorescence emission.
providing a transmissive sensor positionable to sense the presence of at least a portion of the blood-borne indicator flowing within a blood vessel at arterial vasculature of one or more symmetrically paired distal locations of the patient and having one or more outputs corresponding with the instantaneous concentration of indicator at such blood vessel vasculature;
providing a monitor/controller having a display and responsive to said actuation to commence measuring the relative concentration of blood concentration of a liver activity indicator in the blood, responsive to a sensor output to display one or more metrics reflecting indicator concentrations to determine the nature of liver function.

2. The system of claim 1 wherein the skin surface is one or more of the ear, the finger of the hand, the neck, the leg, or the arm.

3. The system of claim 1 wherein the skin surface is at paired distal location comprising ears, paired fingers, paired locations on the hands, paired toes, paired locations on the feet, paired locations on the legs, or paired locations on the arms.

4. The system of claim 3 wherein the paired distal location is the ears.

5. The system of claim 1 in which the monitor/controller publishes the relative indicator concentration decay as a function of time curves and the derived exponential decay coefficient, plasma disappearance rate, and residual relative concentration at 15 minutes after injection of the indicator.

6. The system of claim 1 in which information is desired concerning a patient concerning a medical condition comprising one or more of poisoning, trauma, sepsis, pre-operative screening, post-operative recovery, cirrhosis, hepatitis or organ transplant.

7. The system of claim 1 in which the liver activity indicator analyte is indocyanine green dye.

8. The system of claim 1 in which the sensor further comprises a sensor array with transmission mode sensing in which the sensor array comprises two or more pairs of emitters of excitation photons and fluorescence detectors of the fluorescent analyte of liver activity

9. The system of claim 8 further comprising said photodetectors optionally energizable in a sequence of such emitter detector pairs or energizable simultaneously, wherein the monitor/controller is responsive to elect one or more of that pair exhibiting an average detection signal output of highest intensity.

10. The system of claim 1 in which the light path of excitation photons is arranged with an aspheric collimating lens, and the light path of emitted fluorescent photons to the photodetector is arranged with a collimator plate and an interference filter.

11. The system of claim 1 wherein the excitation source emits photons in a wavelength range of from about 750 to 820 nanometers.

12. The system of claim 8 in which the sensor array excitation source emits photons at a wavelength of from about 780 to 790 nanometers.

13. The system of claim 3 whereby the sensor array positionable at paired distal locations further comprises two fluorescence sensing array fixtures with sensing array arms, removeably attached to a headband.

14. The system of claim 1 wherein the apparatus of the system is configurable for use with a subject that is a laboratory animal, a zoological specimen, a cat, a dog, an elephant, an ape, a horse, or a human.

15. The system of claim 1 in which:
the indicator delivery assembly comprises a flexible elongate delivery tube extending between proximal and distal ends, an auxiliary catheter coupled in fluid transfer relationship with the distal end defining the outlet, a indicator flow initiation detector coupled in fluid transfer relationship with the proximal end, and a three-way valve connected upstream to the sensor, a first indicator containing syringe coupled in indicator flow relationship with the valve and actuateable to cause indicator to flow through the valve, and a second isotonic saline fluid containing syringe coupled in fluid flow relationship with the valve and actuateable to cause isotonic saline to flow through the valve.

16. A system for measuring a relative organ activity in a patient, comprising the steps:
providing an indicator analyte delivery system having an outlet located in a vein of the patient in blood flow communication with the right side of the heart;
said indicator analyte delivery system being actuateable to cue the injection of a fluorescing biocompatible dye excitable by tissue penetrating excitation radiation to derive fluorescence emission corresponding with the indicator analyte concentration;
the indicator delivery system includes a flow sensor responsive to derive signals corresponding with the commencement and termination of fluid flow through the system;
providing an indicator analyte that is fluorescently labeled and the concentration of said indicator analyte is responsive to the metabolic status of said organ;
a sensor array comprising a excitation photon emitter energizable to generate light at the excitation radiation wavelength and a photodetector which is filtered for response substantially only to the fluorescence emission.
providing a transmissive sensor positionable to sense the presence of at least a portion of the indicator at the vasculature of one or of symmetrically paired distal locations of the patient and having one or more outputs corresponding with the instantaneous concentration of indicator at such vasculature in which the sensor array further two or more paired excitation photons emitters and photodetectors and energizable in a sequence of such pairs or simultaneously;
the sensor array further comprising the excitation photon emitters and photodetectors arranged with filtering system comprising an aspheric collimating lens, a collimator plate and an interference filter in the transmission path to the photodetector
providing a monitor/controller having a display and responsive to said actuation of the indicator analyte delivery system to commence timing the time following first detection of indicator analyte, responsive to a sensor output; and
providing an associated monitor/controller responsive to publish one or more of a decay curves or to display one or more indicator analyte decay curves to determine relative activity of the organ, whereby the injection of the indicator analyte commences activation of the excitation photon emitters, and detection of any data signal by the associated photodetectors, with the monitor controller collecting said data signal and calculating a organ function metric.

17. The system of claim 16 wherein the organ function metric is one or more of the relative indicator concentration decay as a function of time curves, the derived exponential decay coefficient, plasma disappearance rate, and residual relative concentration at 15 minutes after injection of the indicator.

18. The system of claim 16 wherein the paired distal location comprises the skin surface at ears, paired fingers, paired locations on the hands, paired toes, paired locations on the feet, paired locations on the legs, or paired locations on the arms.

19. The system of claim 16 in which said organ activity is the activity of an organ which is one or more of liver, kidney, endocrine gland, brain, pancreas, lungs, or heart.

20. A sensing array apparatus comprising
(a) a plurality of sources of excitation photons and photodetector pairs for measuring the fluorescence emitted by a fluorescent moiety of a liver activity indicator analyte;
b) said sources of excitation photons providing an excitation light source emitting a first wavelength for excitation of a liver activity indicator within the tissues of a patient body, said emitters transmitting the excitation light through a collimator lens having a collimating channel aligned with an optical path interference filter, said collimating channel and interference filter located intermediate between said sources of excitation photons and photodetector; and
(c) said photodetectors providing for measuring the intensity of the fluorescent light emitted at a second wavelength from the fluorescent moiety of the liver activity indicator analyte; and,
(d) a support system of a plurality of array support arms;
wherein the array support arms of the support system allow positioning of the sensing array apparatus on the exterior of the patient body, whereupon activation of one or more of the sources of excitation photons transmit excitation light through tissue of the patient, thereby exciting liver activity indicator analyte present in said tissue, said photodetectors thereby measuring the intensity of light emitted by excited liver activity indicator analyte.

21. The sensing array apparatus of claim 20 wherein the plurality of sources of excitation photons and photodetector pairs are three laser diode emitter and photodetector pairs.

22. The system of claim 20 wherein said sensor assembly further comprises:
a first collimator having a collimating channel aligned with an optical path and located intermediate the other surface of the scaphoid fossa of the ear and an interference filter, and a second collimator having a collimating channel aligned with an optical path and located between the interference filter and the photodetector.

23. The system of claim 20 in which:
the array support arms further comprise a first and a second branch respectively of an assembly supporting the source of excitation photons and of an assembly supporting the photodetectors; and
said first and second branches are joined together in close association to form a fixed throat.

24. The system of claim 14 further comprising a tissue that is circulating blood, plasma or lymph.

25. A kit supplying consumable materials necessary for quantifying liver function comprising:
a) an indicator delivery tubing system providing a valve, syringe connectors, a flow sensor and sterile intravenous injector;
b) one or more doses of liver activity indicator reagent as a shelf stable material;
c) a diluent for preparing the dose of liver activity indicator reagent for injection or for delivering an indicator bolus; and
d) a dose of nonreactive blood compatible clearing reagent for completing the injection.

26. The kit of claim 25 further comprising a flow initiation sensor that further comprises an initiation sensor with a circuit that in communication with a monitor-controller responds to a query determining the number of injections a flow initiation sensor has cued and that is disabled for repeat use after a testing procedure time period.

27. The kit of claim 25 further comprising a sealed tray containing the kit contents maintained in a sterile condition until opened.

28. A method for measuring the relative concentration over time of a liver activity indicator in the circulating blood stream, comprising the steps:
a) selecting a liver activity indicator analyte that is predominantly removed from circulating blood by the liver said analyte further comprising a fluorescent moiety that can be excited to emit fluorescence through irradiation by excitation photons in wavelength range known to induce fluorescence in said analyte;
b) positioning about the skin of a human subject one or more emitter/detector arrays for emitting excitation photons in a given wavelength range known to induce fluorescence in said fluorescent moiety of the liver activity indicator analyte, said emitter in alignment with one or more detectors configured to measure the intensity of emitted fluorescence photons from the liver activity indicator analyte;
c) injecting said liver activity indicator analyte into the blood stream of a human subject;
d) recording the relative concentration over time of the liver activity indicator analyte by recording time-varying fluorescence signal level; and
e) calculating a liver activity metric according to Formula 1, whereby the relative liver activity of the patient is displayed in a report.

29. The method of claim 28 wherein one or more emitter sources and one or more detectors are positioned opposite one another, each on one side of the ear, finger, toe, tongue, nasal labium, lip or cheek and said detectors aligned with the excitation photons emitted by the emitter source.

30. The method of claim 29 wherein the pairs of emitter sources and detectors are positioned at paired distal locations comprising both the left and right ear, left and right finger, left and right toe, left and right side of tongue, left and right side of lip or left and right cheek.

31. The method of claim 28 wherein the liver activity indicator analyte is indocyanine green dye or infracyanine green dye.

32. The method of claim 28 wherein the wavelength range of the one or more excitation sources is from about 750 to 820 nanometers.

33. The method of claim 32 wherein the wavelength range of the one or more excitation sources is from about 770 to 780 nanometers.

34. The method of claim 1 wherein the recording of the relative concentration of the liver activity indicator analyte over time begins not less than 2.0 minutes after the time of injection of the liver activity indicator into the patients blood stream and continues for at least 15.0 minutes after the time of injection of the liver activity indicator into the patients blood stream, wherein the frequency of recording the relative concentration liver activity indicator analyte is at least once a minute.

35. The method of claim 34 wherein the recording of the relative concentration of the liver activity indicator analyte over time begins not less than 2.0 minutes after the time of injection of the liver activity indicator into the patients blood stream and continues for about 15.0 minutes.
